(19) United States
(12) Patent Application Publication
(10) Pub. No.: US 2025/0129437 A1
(43) Pub. Date: Apr. 24, 2025

(54) ANALYSIS OF MICROBIAL DNA FOR DISEASE CLASSIFICATION

(71) Applicant: Centre for Novostics, Shatin (HK)

(72) Inventors: Yuk-Ming Dennis Lo, Hmantin (CN); Kwan Chee Chan, Jordan (CN); Wai Kei Lam, Kowloon (CN); Peiyong Jiang, Pak Shek Kok (CN); Guangya Wang, Shatin (CN)

(21) Appl. No.: 18/926,028

(22) Filed: Oct. 24, 2024

Related U.S. Application Data
(60) Provisional application No. 63/545,540, filed on Oct. 24, 2023.

(51) Int. Cl. C12Q 1/689

(52) U.S. Cl. CPC C12Q 1/689; C12Q 2600/156

(57) ABSTRACT

A level of a particular microbial disease in the biological sample of a subject is determined. In one example, an amount of cell-free DNA molecules corresponding to the particular microbial species associated with the particular microbial disease is determined using a masked microbial reference genome. The masking can remove regions that are shared with another species. In another example technique, end motifs of cell-free DNA fragments from the subject and from the particular microbial species are used. A correlation can be determined between the amounts of a set of end sequence motifs for the subject and the particular microbial species. For TB, the two sets of amounts are substantially more correlated for a positive subject than for a negative subject.

### CROSS-REFERENCES TO RELATED APPLICATIONS

The present application claims priority from and is a non-provisional application of U.S. Provisional Application No. 63/545,540, entitled “Analysis of Microbial DNA For Disease Classification” filed Oct. 24, 2023, the entire contents of which are herein incorporated by reference for all purposes.

### BACKGROUND

Pleural fluid refers to the liquid collection that is located between the two layers of the pleura. In a healthy human individual, the pleural space contains a small amount of fluid (about 10 to 20 mL), which contains low levels of white blood cells, proteins and nucleic acids. Pleural effusion refers to the excessive accumulation of fluid in the pleural space, which could be caused by infection, malignancy, or inflammatory conditions.

Tuberculosis (TB) remains one of the major infectious diseases causing millions of deaths each year. Tuberculosis is caused by the infection of a group of Mycobacterium species (Mycobacterium tuberculosis complex, MTBC). They are characterized by over 99% similarity at the nucleotide level and identical 16S rRNA sequences to the representative pathogenic species Mycobacterium tuberculosis (MTB) (Brosch et al. Int J Med Microbiol. 2000; 290 (2): 143-52). The mycobacteria not included in MTBC and Mycobacterium leprae are known as nontuberculous mycobacteria (NTM).

TB diagnostics remains a challenge in the disease management because of the difficulty in the culture of Mycobacterium. Microbiological culture by using sputum or other bodily fluids have long been used as a gold standard for TB diagnostics, but the time to positive detection could take several weeks, which fails to meet the need for prompt TB diagnosis and treatment (Moore et al. Diagn Microbiol Infect Dis. 2005; 52 (3): 247-54; Chang et al. Sci Rep. 2022; 12 (1): 16972). To address this issue, molecular diagnostic methodology (e.g., Xpert MTB/RIF) for detection of mycobacterial DNA, which provides rapid testing results, has been proposed (Ismail et al. PLOS One. 2015; 10 (11):e0141851). However, this method has suboptimal sensitivity (about 50-70%) due to low levels of mycobacterial nucleic acids in specimens (Yu et al. PLOS One. 2021; 16 (6):e0253658; Pan et al. PLOS One. 2021; 16 (6):e0253879).

### BRIEF SUMMARY

Methods, systems, and apparatuses are provided for determining a level of a particular microbial disease in the biological sample of a subject. The biological sample can include cell-free DNA of bacteria and cell-free DNA of the subject.

In one example technique, an amount of cell-free DNA molecules corresponding to the particular microbial species, which is associated with the particular microbial disease, can be determined using a masked microbial reference genome. The masking can remove regions that are shared with one or more other species. Such masking can filter out DNA molecules that are falsely identified as being from the particular microbial species, which increases accuracy in determining the level of the disease.

In another example technique, end motifs of cell-free DNA fragments from the subject and from the particular microbial species are used. A correlation can be determined between the amounts of a set of end sequence motifs for the subject and the particular microbial species. For TB, the two sets of amounts are substantially more correlated for a positive subject than for a negative subject.

One general aspect includes a method of analyzing a biological sample to determine a level of a particular microbial disease in the biological sample of a subject. The method can include analyzing cell-free DNA molecules from the biological sample to obtain sequence reads. A masked microbial reference genome of a particular microbial species that is associated with the particular microbial disease can be stored. The masked microbial reference genome can be generated from a microbial reference genome of the particular microbial species. The microbial reference genome can include (1) specific regions that are identified as unique to the particular microbial species and (2) non-specific genomic regions that are shared with one or more other species. The masked microbial reference genome can be generated by removing the non-specific genomic regions from the microbial reference genome. The method can also include aligning the sequence reads to the masked microbial reference genome to identify a group of the cell-free DNA molecules as being from the particular microbial species. The method can also include determining an amount of the group of the cell-free DNA molecules. The method can also include determining a classification of the level of the particular microbial disease for the subject based on a comparison of the amount to a reference value.

Another general aspect includes a method of analyzing a biological sample to determine a level of a particular microbial disease in the biological sample of a subject. The method can include analyzing cell-free DNA molecules from the biological sample to obtain sequence reads. Analyzing a cell-free DNA molecule can include determining an end sequence motif of at least one end of the cell-free DNA molecule. The method can also include identifying, by comparing the sequence reads to a human reference genome, a first group of the cell-free DNA molecules as being from the subject. The method can also include identifying, by comparing the sequence reads to a microbial reference genome, a second group of the cell-free DNA molecules as being from a particular microbial species that is associated with the particular microbial disease. The method can also include determining, using the sequence reads of the first group of the cell-free DNA molecules, a first amount for each of a set of end sequence motifs of the first group of the cell-free DNA molecules, thereby obtaining first amounts. The method can also include determining, using the sequence reads of the second group of the cell-free DNA molecules, a second amount for each of the set of end sequence motifs of the second group of the cell-free DNA molecules, thereby obtaining second amounts. The method can also include measuring a correlation value of a correlation between the first amounts and the second amounts. The method can also include determining a classification of the level of the particular microbial disease for the subject based on a comparison of the correlation value to a reference value.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 is an example illustration of lung and chest cavity of a patient having pleura effusion.

FIG. 2 shows an illustration of Mycobacterium tuberculosis complex (MTBC) and mycobacterial species.

FIG. 3 shows a summary of clinical information of experimental samples and subjects.

FIG. 4 shows example MTB whole genome capture probes.

FIG. 5A shows a number of MTBC-derived DNA fragments in pleural fluid samples.

FIG. 5B shows normalized MTBC abundance of MTBC-derived DNA fragments in pleural fluid samples.

FIG. 6 shows a number of DNA fragments in pleural fluid samples aligned to TB and non-TB species using Bowtie.

FIG. 7 shows a number of DNA fragments classified as from MTBC, unclassified Mycobacterium genus and nontuberculous mycobacterial genera (NTM). FIG. 7 also shows a log-scale number of DNA fragments classified as from MTBC.

FIG. 8A shows an ROC curve using abundance of MTBC in countries where mycobacterial is not endemic. FIG. 8B shows an ROC curve using abundance of MTBC in countries where mycobacterial is endemic.

FIG. 9 illustrates a first method for MTB reference genome masking.

FIG. 10 shows a second method for producing a masked MTB reference genome.

FIG. 11A shows the results using the Kraken2 software for the larger set of samples: 23 TB and 55 non-TB. FIG. 11B shows the results using the masked MTB genome.

FIG. 12 is a flowchart illustrating a method for analyzing a biological sample to determine a level of a particular microbial disease in the biological sample of a subject according to embodiments of the present disclosure.

FIGS. 13A-13B show fragment size distribution of human nuclear DNA in plasma and pleural fluid according to embodiments of the present disclosure. FIG. 13A shows the fragment size distribution of human nuclear DNA in plasma (blue) and pleural fluid (red). FIG. 13B shows the cumulative frequencies of human nuclear DNA size in plasma (blue) and pleural fluid (red).

FIG. 14 shows motif rankings of 4-mer end motifs in plasma and pleural fluid from a patient.

FIG. 15A shows 4-mer end motif rankings for plasma nuclear DNA of two patients. FIG. 15B shows 4-mer end motif rankings for pleural fluid nuclear DNA of two patients. FIG. 16A shows motif O/E ratios for plasma nuclear DNA of two patients. FIG. 16B shows motif O/E ratios for pleural fluid nuclear DNA of two patients.

FIG. 17A shows a correlation matrix representing the correlation coefficients for plasma samples. FIG. 17B shows a correlation matrix representing the correlation coefficients for pleural fluid samples.

FIG. 18 shows a comparison of correlation coefficients for plasma and pleural fluid samples according to embodiments of the present disclosure.

FIG. 19A shows a correlation between human nuclear DNA and MTBC DNA in terms of O/E ratio of 4-mer end motifs for a TB-positive patient. FIG. 19B shows a correlation between human nuclear DNA and MTBC DNA in terms of O/E ratio of 4-mer end motifs (excluding CGNN motifs) for a TB-positive patient.

FIG. 20A shows a correlation between human nuclear DNA and MTBC DNA in terms of O/E ratio of 2-mer end motifs (CG motifs excluded) for a TB-positive patient. FIG. 20B shows a correlation between human nuclear DNA and MTBC DNA in terms of O/E ratio of 2-mer end motifs (CG motifs excluded) for a TB-negative patient.

FIG. 21A is a 2-dimensional plot illustrating the correlation coefficient (between human nuclear DNA and MTBC DNA) and MTBC abundance in pleural fluid samples. FIG. 21B is a ROC analysis showing the performance of abundance and correlation coefficient in distinguishing TB samples from non-TB samples.

FIG. 22A shows a correlation between human nuclear DNA and MTBC DNA for a TB-positive patient in terms of frequency of 2-mer end motifs (CG excluded). FIG. 22B shows a correlation between human nuclear DNA and MTBC DNA for a TB-negative patient in terms of frequency of 2-mer end motifs (CG excluded).

FIG. 23A shows a comparison of correlation coefficients of frequencies between TB and non-TB group samples. FIG. 23B shows a comparison of correlation coefficients of O/E ratios between TB and non-TB group samples.

FIG. 24A shows a principal components analysis (PCA) of 2-mer end motif O/E ratio (excluding CG motifs) of MTBC DNA. FIG. 24B shows a principal components analysis (PCA) of 2-mer end motif frequency (excluding CG motifs) of MTBC DNA.

FIG. 25 provides ROC curves showing the performance of machine learning models trained on MTBC 2-mer motif frequency or motif O/E ratio in distinguishing TB samples from non-TB samples.

FIGS. 26A-26C illustrate fragment size distribution of human nuclear DNA (blue) and MTBC (red) in TB samples.

FIG. 27 is a flowchart illustrating a method for analyzing a biological sample to determine a level of a particular microbial disease in the biological sample of a subject according to embodiments of the present disclosure.

FIG. 28A shows a plot of the number of MTBC fragments determined using nanopore sequencing without masking the MTB reference genome for TB and non-TB subjects. FIG. 28B shows a plot of the number of MTBC fragments determined using nanopore sequencing and masking the MTB reference genome for TB and non-TB subjects.

FIGS. 29A-29F show the comparison of Nanopore and Illumina in terms of size and end motif analysis.

FIG. 30 illustrates a measurement system according to an embodiment of the present disclosure.

FIG. 31 shows a block diagram of an example computer system usable with systems and methods according to embodiments of the present disclosure.

### Terms

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells.

A “biological sample” refers to any sample that is taken from a subject (e.g., a human (or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. Any amount described herein can be any of the numbers listed above. Examples sizes of a sample can include 30, 50, 100, 200, 300, 500, 1,000, 5,000, or 10,000 or more nanograms, or 1, 2, 3, 4, 5, 6, 7, 8, 9, or 10 ml.

The terms “control”, “control sample”, “background sample,” “reference”, “reference sample”, “normal”, and “normal sample” may be interchangeably used to generally describe a sample that does not have a particular condition or is otherwise healthy. In an example, a no-template control (NTC) sample with contaminant DNA can be considered as a reference sample. In another example, the reference sample is a sample taken from a subject without an infection. A reference sample may be obtained from the subject, or from a database. The reference generally refers to a reference genome that is used to map sequence reads obtained from sequencing a sample from the subject. A reference genome generally refers to a haploid or diploid genome to which sequence reads from the biological sample can be aligned and compared. For a haploid genome, there is only one nucleotide at each locus. For a diploid genome, heterozygous loci can be identified, with such a locus having two alleles, where either allele can allow a match for alignment to the locus. A reference genome can be a reference microbe genome that corresponds to a particular microbe species, e.g., by including one or more microbe genomes.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

The phrase “healthy,” as used herein, generally refers to a subject possessing good health. Such a subject demonstrates an absence of any malignant or non-malignant disease. A “healthy individual” may have other diseases or conditions, unrelated to the condition being assayed, that may normally not be considered “healthy”.

The term “fragment” (e.g., a DNA or an RNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. A tumor-derived nucleic acid can refer to any nucleic acid released from a tumor cell, including pathogen nucleic acids from pathogens in a tumor cell. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

The term “infection-causing pathogen-derived microbial DNA” refers to DNA molecules originating from one or more species of microbes known to cause infection in organisms (e.g., humans).

A sequence read can include an “ending sequence” associated with an end of a fragment. The ending sequence can correspond to the outermost N bases of the fragment, e.g., 1-30 bases at the end of the fragment. If a sequence read corresponds to an entire fragment, then the sequence read can include two ending sequences. When paired-end sequencing provides two sequence reads that correspond to the ends of the fragments, each sequence read can include one ending sequence.

A “sequence motif” may refer to a short, recurring pattern of bases in DNA fragments (e.g., cell-free DNA fragments). A sequence motif can occur at an end of a fragment, and thus be part of or include an ending sequence. An “end motif” (also referred to as a “end sequence motif”) can refer to a sequence motif for an ending sequence that preferentially occurs at ends of DNA fragments, potentially for a particular type of tissue. An end motif may also occur just before or just after ends of a fragment, thereby still corresponding to an ending sequence. A nuclease can have a specific cutting preference for a particular end motif, as well as a second most preferred cutting preference for a second end motif. The number of nucleotides (nt) at the fragment ends used for analysis could be, for example, but not limited to, 1 nt, 2 nt, 3 nt, 4 nt, 5 nt, 6 nt, 7 nt, 8 nt, 9 nt, and 10 nt or above. In some embodiments, the fragment end motif could be defined by one or more nucleotides across positions nearby the end of a fragment. The fragment end motif could be defined by one or more nucleotides in a reference genome surrounding the genomic locus to which the end of a fragment is aligned. Various numbers of motifs can be used, e.g., at least 1, 2, 3, 4, 5, 6, 7, 8, 9, 10, 20, 30, 40, 50 60, 70, 80, 90, 100, 150, 200, 250, or 256 end motifs.

A “sequence motif pair” or “end motif pair” may refer to a pair of end motifs of a particular DNA fragment. For example, a DNA fragment having an A at the 5′ end of one strand and an A at the 5′ end of the other strand can be defined as having a sequence motif pair of A< >A. As another example, a DNA fragment having an A at the 5′ end of one strand and an T at the 3′ end of the same strand can be defined as having a sequence motif pair of A< >T, which would correspond to an A< >A fragment defined using 5′ ends of the two strands. Other lengths of sequence motifs can be used. Different paired combinations of end motifs can be referred to as different types of fragments. End motif pairs may include end motifs that are the same length, e.g., both 1-mers or both 2-mers, but may also include end motifs that are of different lengths, e.g., one end is a 2-mer and the other end is composed of 1-mers. End motif pairs may also include one or more bases past the end of the DNA fragment, e.g., as determined by aligning to a reference genome. Such an instance can use the nomenclature t|A, where T occurs just before a cutting site at the 5′ end, and A occurs after the cutting site.

An “end-motif profile” may refer to the relationship of ending sequences (e.g., 1-30 bases) of cell-free DNA fragments (also just referred to as DNA fragments) in a sample. Various relationships can be provided, e.g., an amount of cell-free DNA fragments with a particular ending sequence (end motif), a relative frequency of cell-free DNA fragments with a particular ending sequence compared to one or more other ending sequences. In some instances, the end-motif profiles are determined using other types of parameters, such as size. For example, the end-motif profile can be provided in various ways that illustrate an amount of cell-free DNA fragments having one or more particular ending sequences for a given size (single length or size range).

A “relative frequency” (also referred to just as “frequency”) may refer to a proportion (e.g., a percentage, fraction, or concentration). In particular, a relative frequency of a particular end motif (e.g., A, CG, TAG, etc.) or end motif pair (e.g., A< >A) can provide a proportion of cell-free DNA fragments that have that end motif or that particular pair end motif pair.

An “expected frequency” of the end motifs can be determined based on the reference sequence within the region for a reference genome, e.g., how many times a particular end motif appears in the region of the reference genome. The exact expected frequency would depend on the sequence of the region and may be normalized, e.g., the size of the region as may be defined as the total number of k-mer end motifs in the region. The expected frequency can provide information about whether the measured frequency is higher than expected, since certain regions may have more CpG sites than other regions.

An “O/E ratio” refers to the ratio of observed to expected frequency of a certain end motif (O/E ratio) can be used for downstream analysis. In the O/E ratio, the O is the observed frequency (i.e., normalized amount) of a particular set of one or more k-mer end motifs. The frequency can be determined via any normalization technique described herein. For example, the observed frequency can be determined as the percentage of fragments having one of the particular set of k-mer end motifs out of all of the k-mer end motifs (e.g., 3-mer end motifs).

The term “sequencing depth” refers to the number of times a locus is covered by a sequence read aligned to the locus. The locus could be as small as a nucleotide, or as large as a chromosome arm, or as large as the entire genome. Sequencing depth can be expressed as 50×, 100×, etc., where “x” refers to the number of times a locus is covered with a sequence read. Sequencing depth can also be applied to multiple loci, or the whole genome, in which case x can refer to the mean number of times the loci or the haploid genome, or the whole genome, respectively, is sequenced. Ultra-deep sequencing can refer to at least 100× in sequencing depth.

A “separation value” corresponds to a difference or a ratio involving two values, e.g., two fractional contributions or two methylation levels. The separation value could be a simple difference or ratio. As examples, a direct ratio of x/y is a separation value, as well as x/(x+y). The separation value can include other factors, e.g., multiplicative factors. As other examples, a difference or ratio of functions of the values can be used, e.g., a difference or ratio of the natural logarithms (In) of the two values. A separation value can include a difference and a ratio. A separation value can be compared to a threshold to determine whether the separation between the two values is statistically significant.

The term “parameter” as used herein means a numerical value that characterizes a quantitative data set and/or a numerical relationship between quantitative data sets. For example, a ratio (or function of a ratio) between a first amount of a first nucleic acid sequence and a second amount of a second nucleic acid sequence is a parameter. The parameter can be used to determine any classification described herein, e.g., with respect to fetal, cancer, or transplant analysis. A “separation value” is an example of a parameter (also called a metric) that provides a measure of a sample that varies between different classifications (states), and thus can be used to determine different classifications.

A “correlation value” is an example of separation value between two sets of values, e.g., between pairs of corresponding values. A set of values can form a vector, which can represent a multidimensional data point. As an example, a correlation value can be an aggregation of a difference between each pair of values. Such a value can be normalized, e.g., by the number of pairs. A correlation coefficient is a type of correlation value, e.g., the Pearson coefficient. The correlation values include but are not limited to Pearson correlation coefficient, Spearman's rank correlation, Phi correlation, Kendall rank correlation, Jaccard similarity, Cosine similarity etc. The determination of a correlation value can be implicit via a use of a machine learning model, e.g., clustering, PCA, SVM, or neural networks. Such models can receive both sets of values and provide a score that is dependent on the correlation between the two sets of values.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

A “level of microbial disease” can refer to the existence, amount, degree, or severity of a disease associated with a subject, as well as the disease's response to treatment. Examples of a microbial disease include tuberculosis (caused by Mycobacterium tuberculosis complex, MTBC) and staph infection (caused by staphylococcus bacteria). The level may be zero. A heathy state of a subject can be considered a classification of no disease. The level of disease may be a number or other indicia, such as symbols, alphabet letters, and colors. The level of disease can be used in various ways. For example, screening can check if disease is present in someone who is not previously known to have disease. Assessment can investigate someone who has been diagnosed with the disease to monitor the progress of the disease over time, study the effectiveness of therapies or to determine the prognosis. In one embodiment, the prognosis can be expressed as the chance of a patient dying, or the chance of the disease progressing after a specific duration or time. Detection can mean ‘screening’ or can mean checking if someone, with suggestive features of the disease (e.g., symptoms or other positive tests), has the disease. The disease can be caused by various types of microbes, including bacteria and other microorganisms. The level can also indicate a type of infection, such as tuberculosis, anthrax, tetanus, leptospirosis, pneumonia, cholera, botulism, and Pseudomonas infection. In some instances, the level of disease refers to a condition relating to an organism's response to microbes, including sepsis, bacteremia, and septicemia.

A “machine learning model” (ML model) can refer to a software module configured to be run on one or more processors to provide a classification or numerical value of a property of one or more samples. An ML model can include various parameters (e.g., for coefficients, weights, thresholds, functional properties of function, such as activation functions). As examples, an ML model can include at least 10, 100, 1,000, 5,000, 10,000, 50,000, 100,000, or one million parameters. An ML model can be generated using sample data (e.g., training samples) to make predictions on test data. Various number of training samples can be used, e.g., at least 10, 100, 1,000, 5,000, 10,000, 50,000, 100,000, or at least 200,000 training samples. One example is an unsupervised learning model. Another example type of model is supervised learning that can be used with embodiments of the present disclosure. Example supervised learning models may include different approaches and algorithms including analytical learning, statistical models, artificial neural network (e.g. including convolutional and/or transformer layers), boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ordinal classification, data pre-processing, handling imbalanced datasets, statistical relational learning, or Proaftn (a multicriteria classification algorithm), or an ensemble of any of these types. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to +10%. The term “about” can refer to +5%.

The term “ROC” or “ROC curve,” as used in the present disclosure, can refer to a receiver operator characteristic curve. A ROC curve can be a graphical representation of the performance of a binary classifier system. For any given method, a ROC curve can be generated by plotting the sensitivity against the specificity at various threshold settings. The sensitivity and specificity of a method for detecting the presence of a tumor in a subject can be determined at various concentrations of tumor-derived DNA in the plasma sample of the subject. Furthermore, provided at least one of three parameters (e.g., sensitivity, specificity, and the threshold setting), a ROC curve can determine the value or expected value for any unknown parameter. The unknown parameter can be determined using a curve fitted to a ROC curve. For example, provided the concentration of tumor-derived DNA in a sample, the expected sensitivity and/or specificity of a test can be determined. The term “AUC” or “ROC-AUC” can refer to the area under a receiver operator characteristic curve. This metric can provide a measure of diagnostic utility of a method, taking into account both the sensitivity and specificity of the method. A ROC-AUC can range from 0.5 to 1.0, where a value closer to 0.5 can indicate a method has limited diagnostic utility (e.g., lower sensitivity and/or specificity) and a value closer to 1.0 indicates the method has greater diagnostic utility (e.g., higher sensitivity and/or specificity). See, e.g., Pepe et al, “Limitations of the Odds Ratio in Gauging the Performance of a Diagnostic, Prognostic, or Screening Marker,” Am. J. Epidemiol 2004, 159 (9): 882-890, which is entirely incorporated herein by reference. Additional approaches for characterizing diagnostic utility include using likelihood functions, odds ratios, information theory, predictive values, calibration (including goodness-of-fit), and reclassification measurements. Examples of the approaches are summarized, e.g., in Cook, “Use and Misuse of the Receiver Operating Characteristic Curve in Risk Prediction,” Circulation 2007, 115:928-935, which is entirely incorporated herein by reference.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range (e.g., range can be greater than or less than specified number), and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

Standard abbreviations may be used, e.g., bp, base pair(s); kb, kilobase(s); pi, picoliter(s); s or see, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Metagenomic next-generation sequencing (mNGS) offers an unbiased approach to detecting a wide range of pathogens in clinical samples and has been proposed for a utility in infectious disease diagnostics (Oreskovic et al. J. Clin. Microbiol. 2021; 59 (8):e0007421; Oreskovic et al. Int. J. Infect. Dis. 2021; 112, 330-337). However, metagenomic sequencing assay for tuberculosis diagnostics is again limited by the low concentration of MTB and the background of contaminating nontuberculous mycobacterial DNA, which might have very similar sequences as the pathogenic MTB (Chang et al. Sci Rep. 2022; 12 (1): 16972). Such problems can also affect other diseases and microbes besides for MTB.

To filter out measurements related to nontuberculous mycobacterial DNA, some embodiments can mask out genomic regions that are shared with other microbes, so that measurements of DNA fragments can be more accurately attributed to the particular target microbe. The shared regions can be determined by comparing reference genomes for the different species, e.g., by partitioning one species reference genome using K-mers and then comparing (aligning) the K-mers to one or more reference genomes of one or more other species.

Additionally or alternatively, fragment end motifs of mycobacterial and host-derived nucleic acids are used to detect a particular microbial disease. A metric determined from fragment end motifs can be used to distinguish pathogen derived signals from environmental contamination or microbial classification errors among similar microbial genomic sequences which further reduces false positive rate while retaining high assay sensitivity. Overall, the innovative approach combining MTB capture probes and nucleic acid end motif analysis provides promising improvement in tuberculosis diagnostics.

In some embodiments, a set of MTB-genome-wide capture probes can be used. The set of MTB-genome-wide capture probes could substantially enrich mycobacterial nucleic acid fragments in samples. Through the novel analysis approach, detecting MTBC-derived sequences in pleural fluid samples from patients with tuberculous pleuritis with high sensitivity is achieved.

### I. LUNG SAMPLES

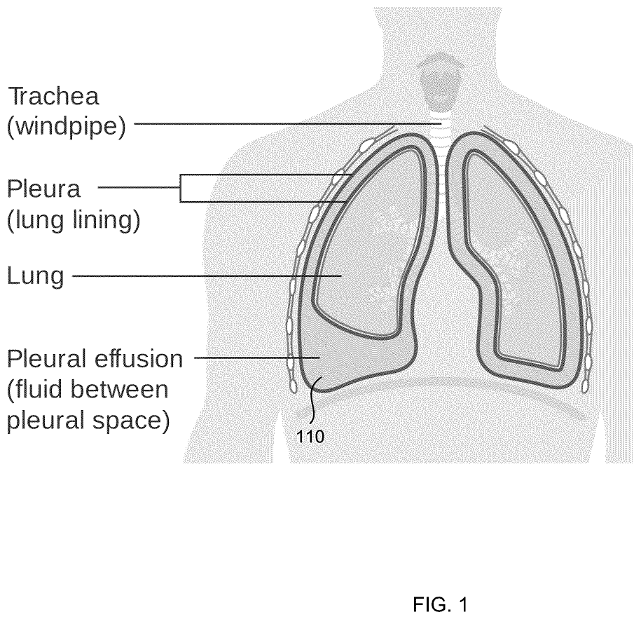

FIG. 1 is an example illustration of lung and chest cavity of a patient having pleura effusion according to embodiments of the present disclosure. Pleural fluid 110 refers to the liquid collection that is located between the two layers of the pleura. In a healthy human individual, the pleural space contains a small amount of fluid (about 10 to 20 mL), which contains low levels of white blood cells, proteins and nucleic acids. Pleural effusion refers to the excessive accumulation of fluid in the pleural space, which could be caused by infection, malignancy, or inflammatory conditions.

Pleural fluid is one example of a biological sample that can be used to determine a classification of a level of the particular microbial disease. Other examples are provided herein, e.g., in the Terms section. For example, sputum, cerebrospinal fluid, urine, and peritoneal dialysate can be used.

### II. MYCOBACTERIUM TUBERCULOSIS COMPLEX (MTBC)

Mycobacteria can cause tuberculosis (TB) but not all mycobacteria cause TB. Thus, populations having a background of nontuberculous mycobacterial DNA can cause difficulties in accurately diagnosing TB. Other microbes and their associated diseases can also have similar problems.

### A. Example Illustration of Mycobacteria Species

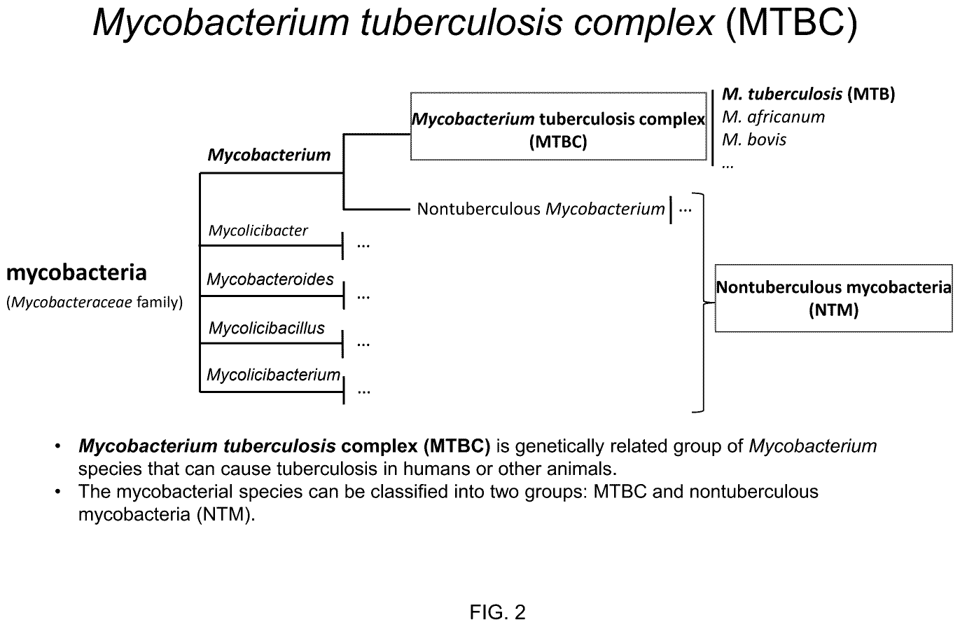

FIG. 2 shows an illustration of Mycobacterium tuberculosis complex (MTBC) and mycobacterial species according to embodiments of the present disclosure. Mycobacterium tuberculosis complex (MTBC) refers to a genetically related group of Mycobacterium species that can cause tuberculosis in humans or other animals. MTBC belongs to the Mycobacteraceae family and has a similar genome sequence as other species that do not cause tuberculosis. Accordingly, the mycobacterial species may be classified into two groups: MTBC and nontuberculous mycobacteria (NTM). Specifically, MTBC are characterized by over 99% similarity at the nucleotide level and identical 16S rRNA sequences to the representative pathogenic species Mycobacterium tuberculosis (MTB). (Brosch et al. Int J Med Microbiol. 2000; 290 (2): 143-52). When an individual has tuberculosis, the bacteriological burden may be at a low level for DNA coming from the MTB bacteria.

### B. Example Techniques

Conventionally, when analyzing the pleural fluid for diagnostics and MTBC, a biopsy of the patient's lung lining is taken for microbiological culture. Microbiological culture uses sputum or other bodily fluids for TB diagnostics, but the time to positive detection could take several weeks, which fails to meet the need for prompt TB diagnosis and treatment. Recently, research has been done in taking pleural fluid and measuring the Mycobacterium tuberculosis DNA, but it has been shown that the sensitivity or the level of the mycobacterial DNA is not high. Additionally, molecular diagnostic methodology (e.g., Xpert MTB/RIF) for detection of mycobacterial DNA has been proposed to provide rapid testing results. However, due to low levels of mycobacterial nucleic acids in specimens, this method has suboptimal sensitivity that can be potentially improved.

### 1. Targeted Approach

To detect and analyze MTBC in pleural fluid, twenty pleural fluid samples from twenty patients with TB infection (TB group) or without TB infection (non-TB groups) were collected. The pleural fluid samples were collected by using a needle penetrating into the patients' pleural fluid and a volume of 10 to 20 mL of the pleural fluid were drawn via the needle. Target sequencing of pleural fluid samples was performed with enrichment of MTB DNA molecules from pleural fluid DNA libraries. In some embodiments, the enrichment was done through the hybridization capture probe system. MTB capture probes were designed to cover the entire bacterial genome. In some embodiments, capture probes that target human autosomal regions were also included in the capture reaction for reference.

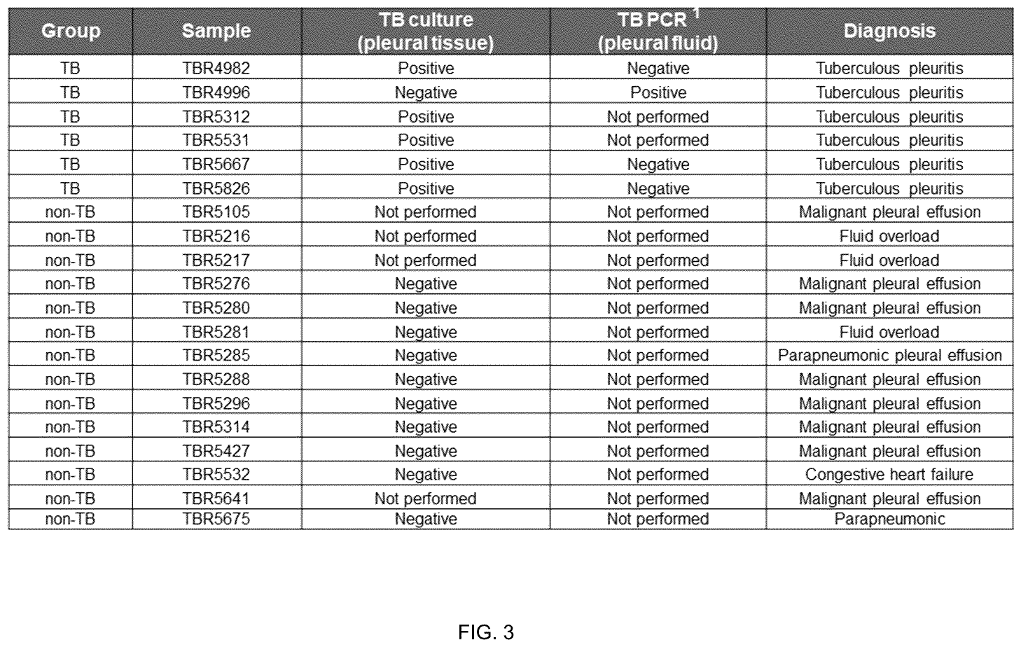

FIG. 3 shows a summary of clinical information of experimental samples and subjects according to embodiments of the present disclosure. Among the twenty patients, six patients having tuberculous pleuritis (TB group) were confirmed with either positive pleural tissue TB culture and/or pleural fluid TB PCR. The fourteen patients without tuberculous pleuritis (non-TB group) were recruited as negative controls.

To address issues related to the low levels of mycobacterial nucleic acids in specimens or low abundance of MTBC DNA in pleural fluid, MTB whole genome capture probes can be used.

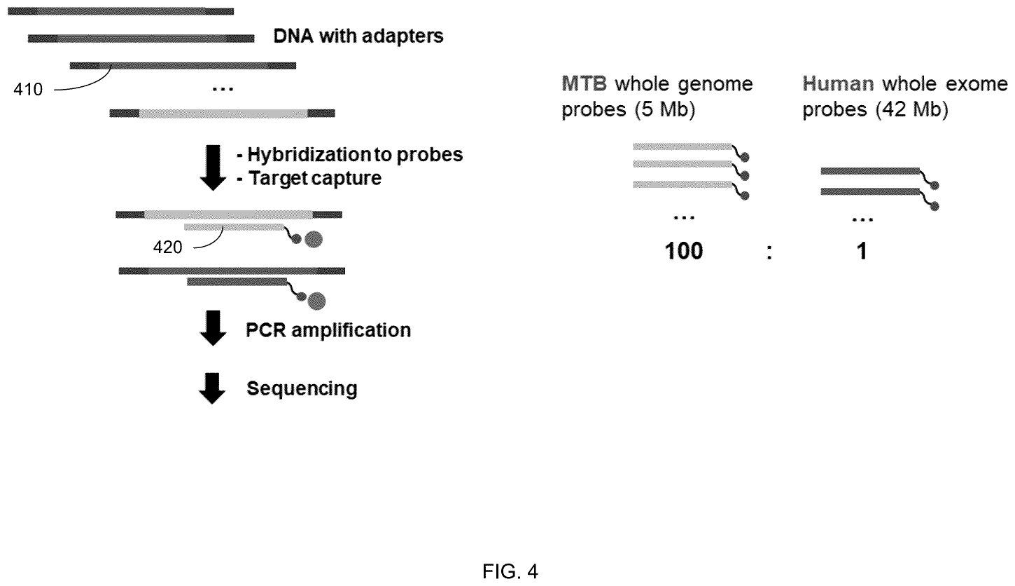

FIG. 4 shows example MTB whole genome capture probes according to embodiments of the present disclosure. The number of probes needed may be determined by dividing the target genome size by the size of the probe (e.g., 5 Mb bp divided by 80 bp). As an example, the probes may include 80-120 base pairs. The probes/primers are used to amplify target MTB genome.

In some embodiments, as sequencing depth can have an impact on the number of the TB reads being detected, to normalize MTBC abundance by the background human DNA, human whole exome target capture probes can be mixed with MTBC probes, e.g., at a specified concentration ratio before experiment. Various concentration ratios can be used, e.g., at least 1000:1, 500:1, 200:1, 100:1, 50:1, or 10:1 where the amount of probes for MTBC being higher than for the human genome.

As illustrated by FIG. 4, DNA fragments 410 with adapters are hybridized to probes 420 for target capture. PCR amplification and/or sequencing can follow as needed. The target capture of the MTB genome can focus on various sizes of the reference genome, e.g., a region having a genome size of approximately 0.5, 1, 2, 3, 4, or 5 Mb. Accordingly, some embodiments may be used to perform target capture sequencing on pleural fluid cfDNA or from other cell-free samples.

In some embodiments, CRISPR-based enrichment strategies (e.g. CRISPR/dCas9-Based Systems) for targeted sequencing can be used.

### 2. Other Techniques

In other embodiments, genomewide or random sequencing can be used. Thus, targeted techniques are not required. Further, for targeted techniques, PCR or other amplification techniques can be used with or without sequencing. For example, digital PCR or real-time PCR can be used for at least some embodiments. Various types of amplification and/or sequencing techniques can be used.

### III. DIFFICULTIES FOR ABUNDANCE OF MYCOBACTERIAL DNA IN PLEURAL Fluid

When non-TB mycobacterial DNA are present, the abundance of MTBC fragments can be inaccurate.

### A. Abundance of MTBC

The abundance of MTBC can be determined by aligning sequence reads to different reference genomes, and the number of fragments that align to the MTBC species can be counted. Various techniques and criteria can be used to determine how to assign sequence reads to a specific species and/or genus. Such techniques include taxonomy techniques such as Kraken, Kraken2, Megablast, Centrifuge, KrakenUniq, and MetaPhlAn. Additionally, alignments tools (e.g., bowtie2, bowtie, bwa, soap, and minimap2) can be used on their own along with a cutoff of a mapping quality.

1. Taxonomic Labels (e.g. Using Kraken2)

Kraken2 is a system for assigning taxonomic labels to short DNA sequences, usually obtained through metagenomic studies. A bioinformatics pipelines can analyze the short DNA sequence from bacteria to determine a genus level and a species level for each sequence read. The taxonomic labels can then be used to determine an abundance of MTBC or other particular microbial species.

The taxonomic classification software can use a database of different microbes (e.g., different reference genomes of different microbes). The reference genomes in the microbial database are from different microbial species. Sequences are aligned at species-level. However, a sequence might be equally aligned to multiple reference genomes of different species under the same genus with the same mapping quality. In this case, the sequence may only be classified and labeled at a genus level.

In another example, a top-down approach can be used to first try to align a sequence read to a reference genome at the genus level, referred to as a genus reference genome. Then, the software can try to align to one or more other reference genomes further down the taxonomy tree, to see whether the read can be mapped to a reference genome of a specific species, referred to as a species reference genome. If the mapping quality improves, then the species may be identified. For example, if the sequence is specific enough, the DNA fragment can be classified into a specific species. However, if the sequence can be aligned into multiple species with the same quality, indicating there is not a specific, unique alignment. In that case, the sequence can be classified to, for example, the genus level or higher.

Accordingly, after targeted capture and sequencing, e.g., as discussed in relation to FIG. 4, DNA fragments can be aligned to reference genome such as MTBC genome and human genome. DNA fragments that were aligned to the MTBC genome were defined as MTBC fragments. DNA fragments aligned to Mycobacterium genus but cannot be assigned to any further level, e.g., species level, were defined as unclassified Mycobacterium fragments. DNA fragments aligned to nontuberculous Mycobacterium species or other nontuberculous genera under Mycobacteriaceae family were referred to as nontuberculous mycobacterial fragments. The number of MTBC DNA fragments could be counted, and further normalized by the number of detected human DNA fragments in the same sample, e.g., MTBC abundance (Reads Per Million Reads, RPM).

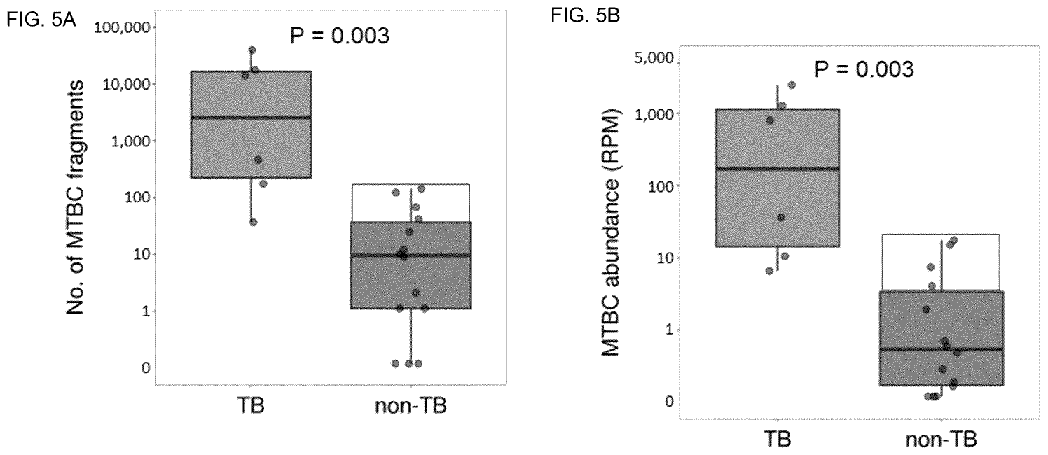

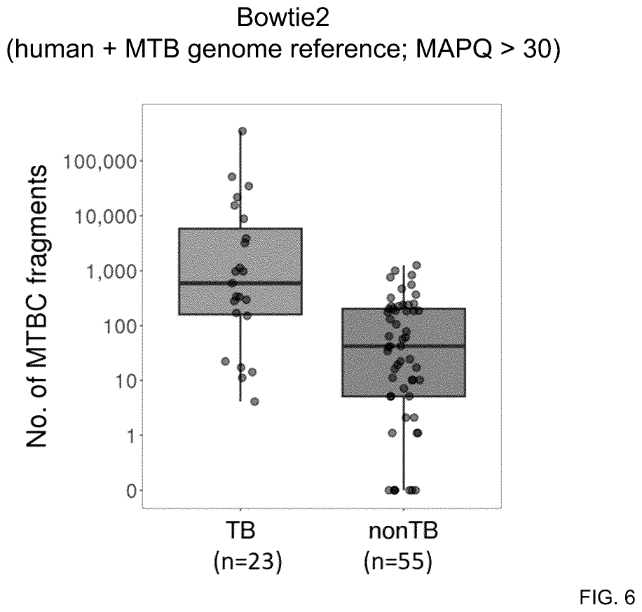

FIG. 5A shows a number of MTBC-derived DNA fragments in pleural fluid samples. FIG. 5B shows normalized MTBC abundance of MTBC-derived DNA fragments in pleural fluid samples. For FIG. 5B, the normalization was done by the number of detected human DNA fragments in the same sample.

The data of FIGS. 5A-5B are based on samples from twenty patients. Among the twenty patients, six patients have TB infection and fourteen patients have no TB infection. As both TB culture and TB PCR results are available, the corresponding results together are used as the gold standard. Commercially available PCR is used to obtain the TB PCR result and a single-region marker is used. As shown in FIGS. 5A-5B, although the MTBC abundance in TB group was generally higher than that in non-TB group, there was an overlap between the two groups that may be potentially caused by the high background non-TB data. As FIG. 5A illustrates, nearly 100 MTBC fragments could be detected in four non-TB group samples.

### 2. Bowtie2

As another example for genus/species classification, the Bowtie alignment tool was used. The sequence reads were aligned to a human reference genome as well as the reference genome of MTBC. The mapping quality was determined, with a cutoff of a mapping quality of 30 being used. Mapping quality is a probability (possibly scaled based on base calling, such as by software Phred) that a read is aligned in the wrong place. Mapping quality of 30 could be equivalent to the alignment error probability of 0.1%. The translation of mapping quality (MQ) to the alignment error rate (E) could be based on the formula: E=10^{-MQ/10}. Various cutoffs, such as 5, 10, 15, 20, 30 or higher could be used. A larger set of samples were analyzing: 23 TB and 55 non-TB.

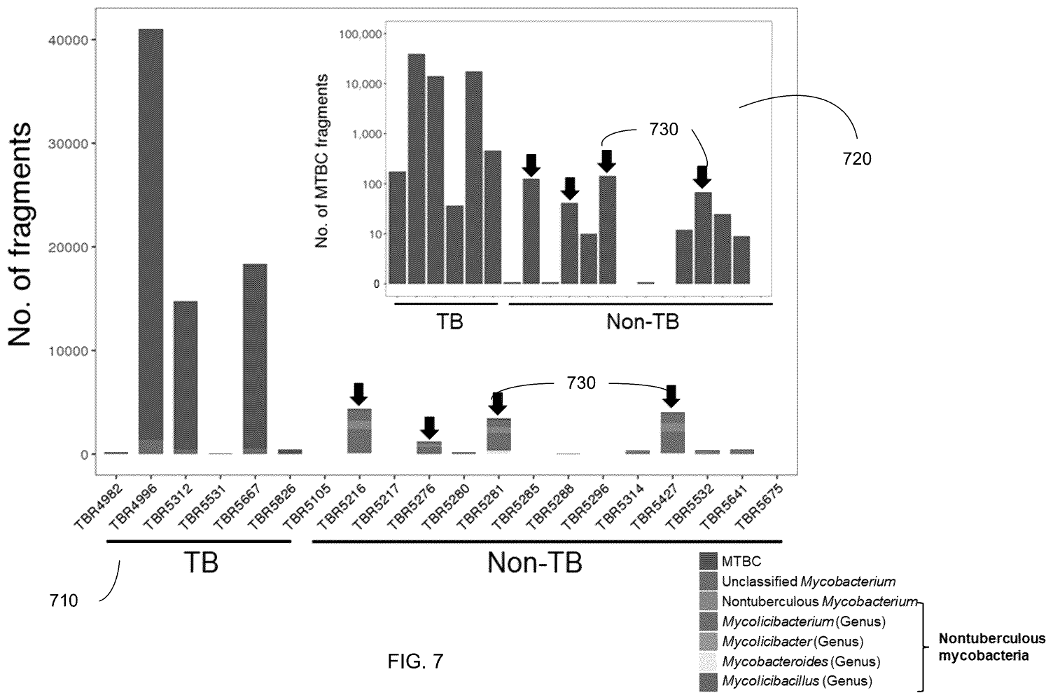

FIG. 6 shows a number of DNA fragments in pleural fluid samples aligned to MTBC and non-MTBC species using Bowtie2. As one can see, there is quite a substantial overlap between the TB cases versus the non-TB cases. The substantial amount of overlap can be attributed to the MTB genome having genetic similarity with the other micro bacteria. Even if the sequence reads could be mapped to the micro bacteria tuberculosis genome with a very high mapping quality, there is a large amount of fragments that are misidentified.

### B. Read Counts Per Subject (MTBC and Nontuberculous Mycobacteria)

The Kraken2 results were further analyzed on a per subject basis for different microbes. The results show that a high background of nontuberculous mycobacterial DNA contamination exists in samples, which might be erroneously classified as MTBC sequences by using the Kraken2 method.

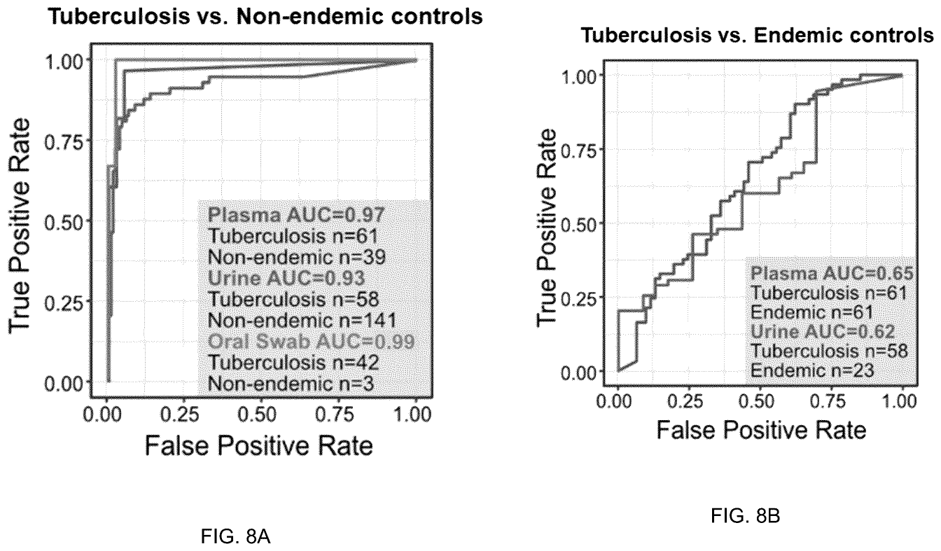

FIG. 7 shows a plot 710 illustrating a number of DNA fragments classified as from MTBC, unclassified Mycobacterium genus and nontuberculous mycobacterial genera (NTM). As discussed earlier, the sequencing reads for the twenty patients can be aligned to the human genome. The reads that cannot be aligned to human genome can then be aligned to reads of a microbial database or effectively microbial reference genomes. The microbial database includes different TB species. Plot 710 shows the number of MTBC fragments detected in each sample without normalization. Plot 710 shows that, in general, the TB samples have more TB rates (i.e., higher number of fragments aligned to the MTBC reference genome).

Plot 710 illustrates the counted number of DNA fragments derived from unclassified Mycobacterium and other non-tuberculous mycobacterial genera. The four non-TB samples with high TB abundance (marked with black arrow 730) tend to have more nontuberculous mycobacterial DNA fragments. The high detection load of MTBC DNA is likely to be caused by high background of nontuberculous mycobacterial DNA contamination in the four samples which were erroneously classified as MTBC sequences due to the high genomic similarity, as indicated in one study (Chang et al. Sci Rep. 2022; 12 (1): 16972). Reads that can be aligned to the Mycobacterium genus but not further to species are shown as “Unclassified Mycobacterium.”

Plot 720 shows a log-scale number of DNA fragments classified as from MTBC. Plot 720 shows normalized MTBC abundance by the number of human reads that can be detected. An overlap can be observed. Similar as plot 710, when compared with non-TB samples, TB samples tend to have a higher abundance MTBC in terms of the number of MTBC fragments.

### A. Non-Endemic Controls and Endemic Controls

In countries where mycobacterial is endemic, the accuracy using abundance of MTBC to detect TB severely decreases, as is shown in Chang et al. Sci Rep. 2022; 12 (1):16972.

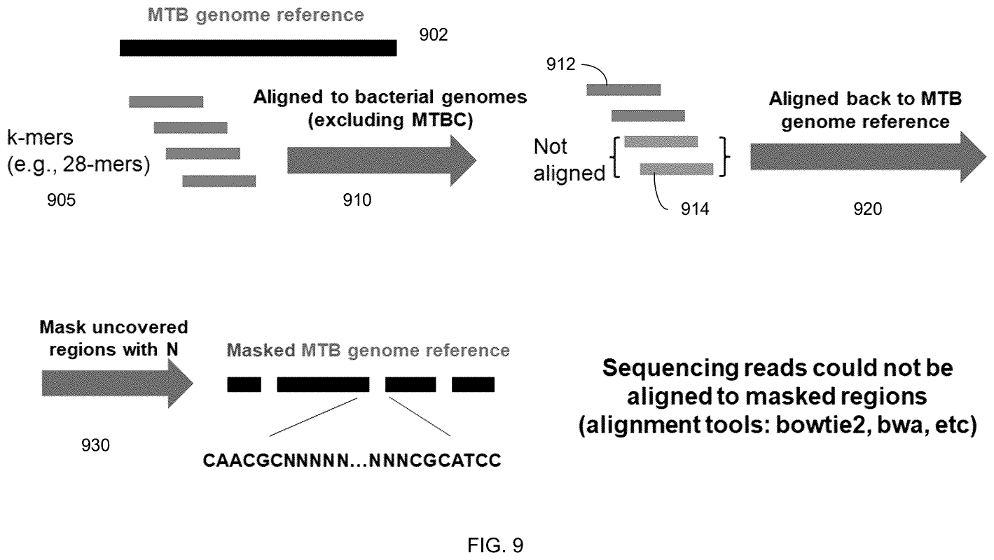

FIG. 8A shows an ROC curve using abundance of MTBC in countries where mycobacterial is not endemic. FIG. 8B shows an ROC curve using abundance of MTBC in countries where mycobacterial is endemic.

As shown in FIG. 8B, nontuberculous mycobacteria background is indistinguishable from a true disease signal due to the low abundance of MTBC and the high sequence similarity between mycobacteria genomes. In the study, plasma, urine, and oral swab samples were collected from patients from the different regions, namely TB endemic and non-endemic regions. Chang performed whole genome sequencing.

Chang concluded that the diagnostics performance is limited by low burden of the Mycobacterium tuberculosis and also the background of nontuberculous mycobacterial DNA. The endemic controls proved to be confounding to the true tuberculosis samples from the endemic regions. The performance was influenced by the background of nontuberculous mycobacterial DNA from the endemic controls.

### IV. MASKING OF SHARED REGIONS IN MTBC

The description above shows the difficulty in differentiating the contaminating reads (e.g., non-TB mycobacterial DNA fragments) when alignment is performed.

### A. Identifying Shared Regions

There is high sequence similarity between Mycobacterium tuberculosis (MTB) genome and genomes of other nontuberculous mycobacteria species or bacterial species from other genera. This could introduce taxonomic classification errors especially when the sequencing reads are short. To tackle this problem, we introduce a masking technique, which can be implemented in various ways. The masking technique can identify and mask non-MTB specific genomic regions, preventing non-MTB reads from being mistakenly classified as from MTB. The masking may be done for any targeted microbial genome for which a background of DNA fragments from similar microbes are to be filtered out.

To identify shared regions, a targeted microbial genome (e.g., MTB reference genome) is compared to background reference genomes (non-targeted microbial genomes). Regions of one genome can be compared to regions of another genome to identify shared regions. Two example methods are described below.

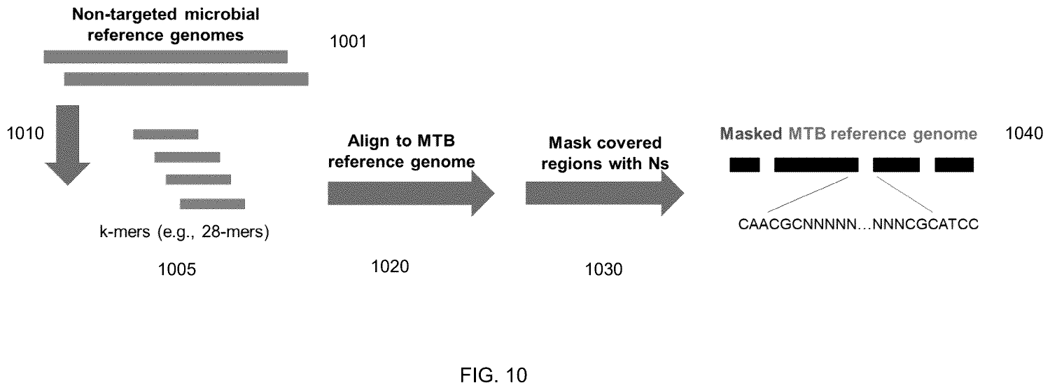

FIG. 9 illustrates a first method for MTB reference genome masking. A set of overlapping K-mers 905 with a length of K are generated from the targeted microbial genome 902 to be analyzed, i.e., MTB. For example, the first method can cut the MTB genome reference into overlapped K-mers, e.g., using sliding windows of length K. Various values for K can be used, e.g., 5, 10, 15, 20, 22, 24, 26, 28, 30, or 32 base pairs. Other values can be used, such as any value in the range 20-35 or lower or higher.

At step 910, these K-mers are aligned to the reference genomes of multiple non-targeted microbial species, e.g., bacterial genomes except for those of Mycobacterium tuberculosis complex. A first set 912 of these K-mers are referred to those that can be aligned to these bacteria genomes, and a second set 914 of the K-mers are those that cannot be aligned to those genomes.

At step 920, the unmapped K-mers (MTB specific K-mers) are aligned back to the MTB reference genome to identify MTB specific regions, or more generally target-specific regions potentially for other applications besides TB.

At step 930, the regions in the MTB reference genome, which are not covered by any K-mers can be masked with “N” characters. Alternatively, the aligned (mapped) K-mers can be aligned back to identify the shared non-specific regions directly.

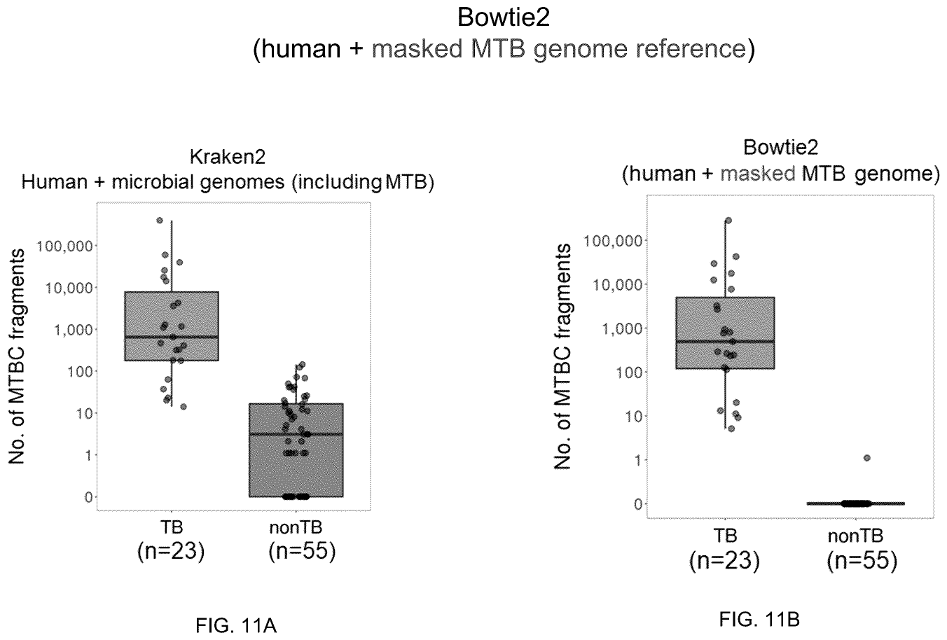

FIG. 10 shows a second method for producing a masked MTB reference genome.

At step 1010, a set of overlapping K-mers 1005 with a length of K are generated from the non-targeted microbial genomes 1001.

At step 1020, these K-mers are aligned to the targeted microbial genome to be analyzed, e.g., MTB. The regions in MTB reference genome that are covered by k-mers are masked with “N” characters (step 1030). Alternatively, the non-aligned (unmapped) K-mers can be aligned back to identify the shared non-specific regions directly.

The masked MTB reference genome 1040 can be used to unambiguously identify the MTB-derived sequences. Confounding sequences are masked out via a high penalty for ambiguous alignment on the masked regions. The alignment tools used here include but are not limited to bowtie2, bowtie, bwa, soap, minimap2, etc. For an alignment tool, if we aligned the reads back to the masked MTB reference genome 1040, the reads cannot be aligned to the regions with N characters, as only MTB specific regions are used or more generally target-specific regions for other microbe/disease targets.

For either method, the target-specific regions can be identified using the aligned or non-aligned K-mers. Depending on whether the K-mers are generated from the target genome or the background non-target genome(s), the aligned reads or the non-aligned reads can be used to identify the target specific regions or the non-target specific regions. If the target specific regions (K-mers) are identified, then the remaining regions (K-mers) can be masked out as being non-target specific regions.

### B. Comparison

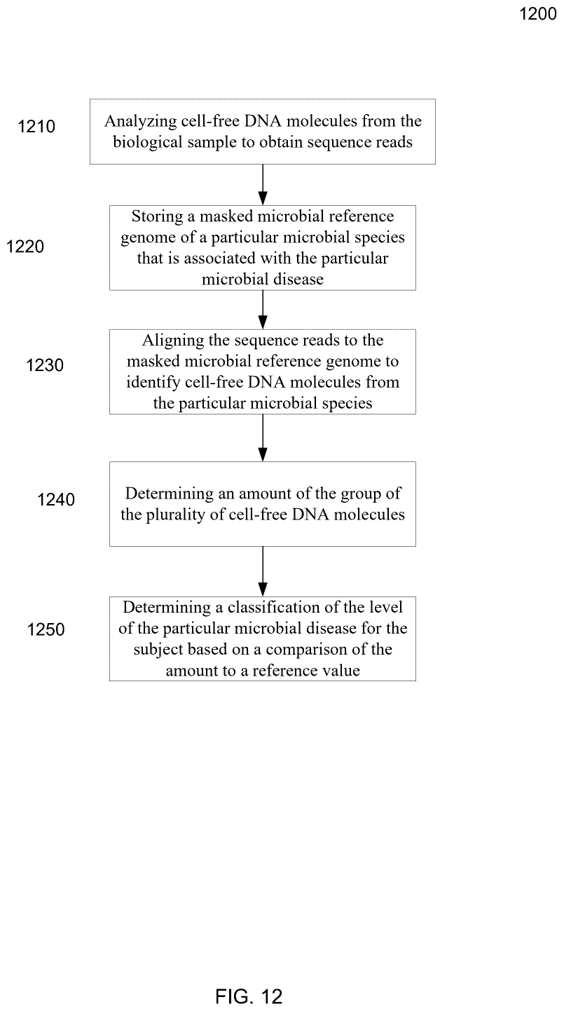

FIG. 11A shows the results using the Kraken2 software for the larger set of samples: 23 TB and 55 non-TB. The overlap in the number of identified MTBC fragments is significant, which is similar to FIG. 5A.

FIG. 11B shows the results using the masked MTB genome. The abundance of MTBC fragments is shown by the number of the MTBC fragments as determined by aligning the sequence reads to MTBC-specific regions using Bowtie2. A mapping quality of 30 or higher was used. Sequences with a mapping quality of 30 or higher were kept. The skilled person will appreciate that other mapping quality values can be used, e.g., depending on a desired sensitivity and specificity. And other alignment software can be used, with corresponding thresholds for mapping quality being determined for the specific alignment software used.

As one can readily see, the separation is much better with only one sample identifying a non-zero (only 1) number of MTBC fragments. Thus, 100% accuracy could be obtained. The Bowtie2 alignment tool was used, but any alignment tool can be used, as will be appreciated by the skilled person. Additionally, the abundance of microbial DNA associated with the particular microbial disease can be normalized

### C. Method Using a Masked Reference Genome

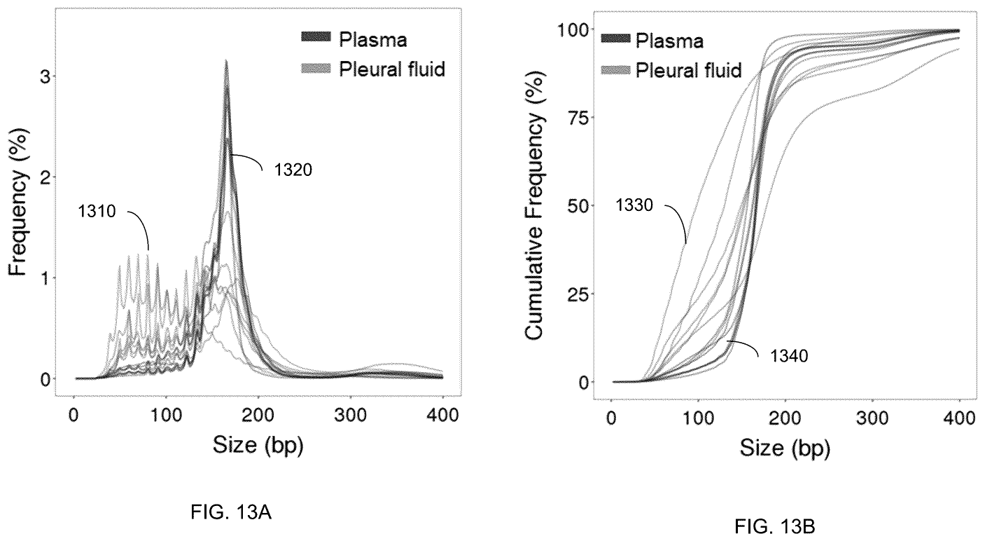

FIG. 12 is a flowchart illustrating a method 1200 for analyzing a biological sample to determine a level of a particular microbial disease in the biological sample of a subject. The particular microbial disease can be associated with a particular microbial species, e.g., a bacterial species. For example, Mycobacterium tuberculosis is associated with TB, and staphylococcus is associated with a staph infection. Method 120 can filter out sequence reads that may be from similar species but not the target microbial species, thereby removing noise and obtaining increased accuracy.

Method 1200 and any of the methods described herein may be totally or partially performed with a computer system including one or more processors, which can be configured to perform the steps. Thus, some embodiments are directed to computer systems configured to perform the steps of any of the methods described herein, potentially with different components performing a respective step or a respective group of steps.

At block 1210, a plurality of cell-free DNA molecules from the biological sample is analyzed to obtain sequence reads. The cell-free DNA molecules can be analyzed by receiving corresponding sequence reads and analyzing the sequence reads by a computer. Various techniques can be used for such analysis in any of the methods described in the present disclosure and may include performing an assay. For example, the analysis can be performed using sequencing, such as massively parallel sequencing, targeted sequencing, and single molecule sequencing (e.g., using a nanopore or using real-time single molecule sequencing (e.g., from Pacific Biosciences)). In some instances, the biological sample is enriched for DNA molecules from the microbes using capture probes that bind to a portion of, or an entire genome of, the microbes. Example PCR techniques include real-time PCR and digital PCR (e.g., droplet digital PCR). The analysis can include the physical steps of performing such assays and receiving of the measurement data obtained from such assays or may just include receiving the measurement data.

In some embodiments, the targeted sequencing can use capture probes for the microbial reference genome that are at a higher concentration than capture probes for the human reference genome, e.g., at ratio described herein.

At block 1220, a masked microbial reference genome of a particular microbial species that is associated with the particular microbial disease is stored. The masked microbial reference genome can be generated from a microbial reference genome of the particular microbial species. The microbial reference genome can include (1) specific regions that are identified as unique to the particular microbial species and (2) non-specific genomic regions that are shared with one or more other species. The specific and non-specific regions can be identified as described herein, e.g., for FIGS. 9 and 10 and corresponding description. The masked microbial reference genome can be generated by removing the non-specific genomic regions from the microbial reference genome. The one or more other species can include the subject and/or other microbes, which may have a similar reference genome, e.g., 10%, 20%, 30%, 40%, 50%, 60%, 70%, 80%, or 90% similar.

At block 1230, the sequence reads are aligned to the masked microbial reference genome to identify a group of the plurality of cell-free DNA molecules as being from the particular microbial species. Any alignment tool may be used (e.g., Bowtie, bwa, etc.) as will be appreciated by the skilled person. When the particular microbial disease is TB, the particular microbial species can be MTBC. Other microbial disease can be associated with another target microbial species.

Aligning a cell-free DNA molecule can include determining a genomic position in a reference genome. For example, one or more sequence reads of a DNA molecule (e.g., paired reads at the ends or a read for the entire molecule) can be aligned or attempted to align to one or more reference genomes (e.g., a target microbial genome and possibly one or more reference genomes of one or more other species) using any of various alignment techniques as will be appreciated by the skilled person. The sequence reads can be aligned to multiple microbial reference genomes in a taxonomy tree to identify the group of the cell-free DNA molecules as being from the particular microbial species, where the multiple microbial reference genomes include the masked microbial reference genome.

The alignment can be to some or all of the masked microbial reference genome. The alignment of the sequence reads to the masked microbial reference genome can use alignment software that outputs a mapping quality. A sequence read is identified as being from the particular microbial species when the mapping quality is greater than a threshold, e.g., 30 or other values described herein.

As another example, probe-based techniques can identify a DNA molecule as being from a particular position, e.g., by emitting a particular color for a particular probe that corresponds to a particular genomic position. The position determination can be to some or all of the reference genome, e.g., if only part of the genome is being analyzed. As examples, the amount of the genome analyzed can be greater than 0.01%, 0.1%, 1%, 5%, 10%, or 50%. Such an analysis may be performed for other methods described herein.

At block 1240, an amount of the group of the plurality of cell-free DNA molecules is determined. The amount can be an absolute amount or be normalized. For example, the amount can be normalized by the total number of reads obtained, e.g., a number of reads per million. Another example is that the normalization uses a number of sequence reads that are identified as being from the subject, e.g., by alignment to a human reference genome. Such reads from the subject can be nuclear or mitochondrial as may be determined using a corresponding reference genome.

At block 1250, a classification of the level of the particular microbial disease for the subject is determined based on a comparison of the amount to a reference value. The reference value can be selected using measurements obtained from one or more reference samples for which a classification is known, e.g., disease positive or disease negative. For instance, the reference value can be determined using a first cohort of training samples from subjects known to have the particular microbial disease and a second cohort of training samples from subjects known to not have the particular microbial disease. FIG. 11B shows a plot with measurements for such reference samples. An example reference value could be 2, 3, 4, or 5, which would provide 100% accuracy for the training set shown in FIG. 11B.

As described above, the one or more non-specific genomic regions can be identified in a variety of ways. For example, method 1200 can identify the non-specific genomic regions that are shared with the one or more other species by comparing the microbial reference genome of the particular microbial species to one or more other reference genomes of the one or more other species. In some implementations, identifying the non-specific genomic regions can include partitioning the one or more other reference genomes into a set of K-mers and aligning the set of K-mers to the microbial reference genome to identify the non-specific genomic regions. As examples, K can be between 20 and 35. The non-specific genomic regions can correspond to a subset of the set of K-mers that aligned to the microbial reference genome. That is, a subset (portion) of the K-mers aligned to the microbial reference genome.

In other implementations, identifying the one or more non-specific genomic regions includes partitioning the microbial reference genome into a set of K-mers and aligning the set of K-mers to the one or more other reference genomes to identify the non-specific genomic regions. As examples, K can be between 20 and 35. In some implementations, the non-specific genomic regions can correspond to a subset of the set of K-mers that aligned to the one or more other reference genomes. In other implementations, the non-specific genomic regions are identified as regions not corresponding to a subset of the set of K-mers that did not align to the one or more other reference genomes. That is, the specific region can be identified and the non-specific regions can be identified as the remaining part of the microbial reference genome.

### V. CORRELATION OF END MOTIFS BETWEEN HOST AND MICROBE DNA

In addition or alternatively to using abundance of DNA fragments from a target microbe genome, end sequence motifs (also referred to as end motifs) can be used. An end motif corresponds to the sequence at either or both ends of a DNA fragments, e.g., a 2-mer at 5′ end of the fragment. Other numbers of bases can be used, and either or both strands can be used. The Terms section provides further elaboration on end sequence motifs.

The description below shows an amount (e.g., a relative frequencies such as rankings) of end motifs of host DNA (e.g., human DNA, nuclear and/or mitochondrial) correlates to target microbial DNA in a sample (e.g., a pleural sample) and can be used to indicate a host has a disease associated with the target microbe.

### A. Fragmentation Analysis of Human DNA in Pleural Fluid and Plasma

The fragmentation end motif signatures of pleural fluid cfDNA have not been studied. Whole genome sequencing (Illumina platform) was performed on 14 paired pleural fluid and plasma samples from 7 patients who have pleural effusions. For each sample, at least 30 million DNA fragments were sequenced.

1. Fragment Size of Pleural Fluid Human Nuclear cfDNA

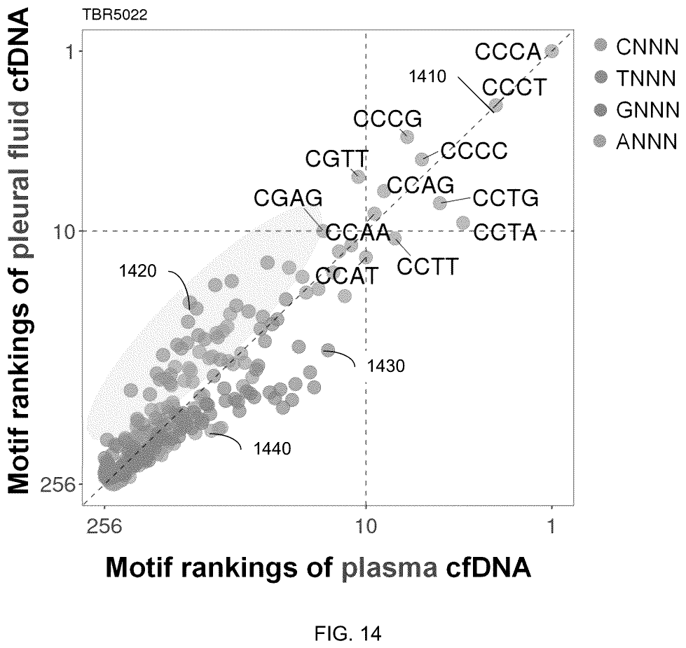

FIGS. 13A-13B show fragment size distribution of human nuclear DNA in plasma and pleural fluid according to embodiments of the present disclosure. FIG. 13A shows the fragment size distribution of human nuclear DNA in plasma 1320 (blue) and pleural fluid 1310 (red). FIG. 13B shows the cumulative frequencies of human nuclear DNA size in plasma 1340 (blue) and pleural fluid 1330 (red).

As shown in FIG. 13A, pleural fluid samples have higher frequency, or fractions, of short cfDNA than plasma samples. Furthermore, pleural fluid samples have lower levels of the 166 bp peak, which is dominant in plasma samples. In particular, the plasma samples show a higher peak of frequency for 100-200 bp. The pleural fluid samples have a more pronounced 10-bp periodicity distribution pattern having frequency peaks before the highest 166 bp. FIG. 13B shows cumulative frequency of human nuclear DNA in plasma and pleural fluid samples. Similar as FIG. 13A, the pleural fluid samples have higher cumulative frequency of short cfDNA fragments than plasma samples.

2. End Motif Analysis of Pleural Fluid Human Nuclear cfDNA

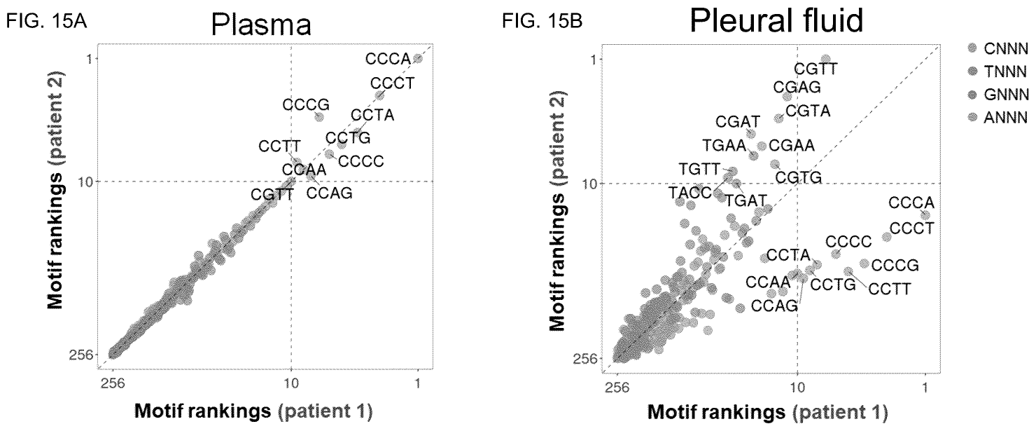

FIG. 14 shows motif rankings of 4-mer end motifs in plasma and pleural fluid from a patient. A ranking uses an amount (e.g., a relative frequency) of DNA fragments having each of a set of end motifs, and then orders (ranks) the end motifs by the amounts. Other example amounts of end motifs can be relative frequencies (e.g., observed frequencies) and O/E ratios of observed to expected frequencies. FIG. 14 uses rankings determined via O/E ratios.

The collection of parameter values (e.g., amount, frequency, O/E ratio, rankings, etc.) of end motifs for a given sample can comprise an end motif profile for that sample. Such end motif profiles can be a vector that represents a multidimensional data point and can be compared, e.g. as shown in FIG. 14 or in other ways. A comparison of end motif profiles can provide a correlation value, such as a distance between the corresponding vectors representing the end motif profiles.

The motif rankings of 4-mer may were determined by analyzing end motif on human nuclear cfDNA in paired plasma and pleural fluid samples. As shown in FIG. 14, the rankings of 4-mer end motifs (256 in total) according to O/E ratios in plasma (x-axis) samples and pleural fluid (y-axis) samples from the same patient were compared. O/E ratios refer to the ratio of observed to expected frequency of a certain end motif (O/E ratio). In the O/E ratio, the O is the observed frequency (e.g., normalized by total amount of reads) of a particular set of end motifs as measured in the sequenced DNA fragments, and the and the E is the expected end motif frequency as determined from reference genome sequences. The frequency can be determined via any normalization technique described herein. For example, the observed frequency can be determined as the percentage of fragments having one of the particular set of k-mer end motifs out of all of the k-mer end motifs (e.g., 3-mer end motifs). An expected frequency of the end motifs can be determined based on the reference sequence within region(s) used for a reference genome, e.g., how many times a particular end motif appears in the region(s) of the reference genome.

The different colors denote the first base of 4-mer motifs. Dots 1410 have C first. Dots 1420 have T first. Dots 1430 have G first. Dots 1440 have A first. N represents A, T, C or G base. For the same patient, pleural fluid and plasma were generally correlated in terms of end motif profiles, but there were still variations. As an example, the T-ends human fragments (e.g., fragments with TNNN end motifs) were preferentially increased in pleural fluid compared with paired plasma samples. In other words, TNNN end motifs are over-presented in the pleural fluid cfDNA in some patients. Previous analysis and studies also show that the DNASE1 may prefer cutting at T-ends, which may indicate a higher concentration of DNASE1 in the pleural fluid relative to plasma.

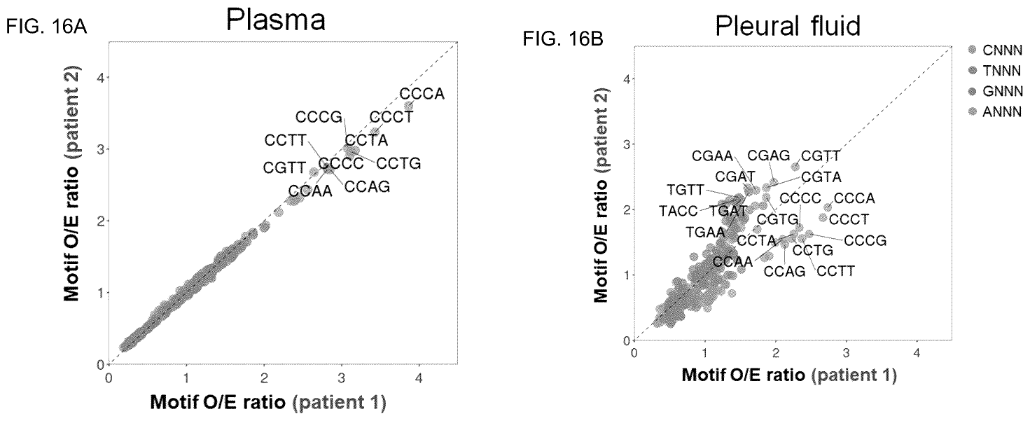

The end motif in multiple plasma and pleural fluid samples were also analyzed. By comparing two plasma samples from different patients, the end motif profiles were highly correlated in terms of motif rankings or O/E ratios (Pearson's r=0.998, FIGS. 15A and 16A). While for paired pleural fluid samples from the same two patients, the correlation decreased (Pearson's r=0.832, FIGS. 15B and 16B).

FIG. 15A shows 4-mer end motif rankings for plasma nuclear DNA of two patients according to embodiments of the present disclosure. The two patients both have medical conditions causing pleural effusion. In particular, x-axis represents the motif rankings of 4-mer end motifs in plasma samples for a first patient, whereas y-axis represents the motif rankings of 4-mer end motifs in plasma samples for a second patient. As FIG. 15A shows, the 4-mer end motif rankings for plasma nuclear DNA of the first patient and the second patient are highly correlated, with some variations between the two patients. The 4-mer end motif rankings for Plasma nuclear DNA of the first patient and the second patient had a Pearson's r of 0.998.

FIG. 15B shows 4-mer end motif rankings for pleural fluid nuclear DNA of two patients according to embodiments of the present disclosure. The two patients both have medical conditions causing pleural effusion. In particular, x-axis represents the motif rankings of 4-mer end motifs in pleural fluid samples for the first patient, whereas y-axis represents the motif rankings of 4-mer end motifs in plasma samples for the second patient. As FIG. 15B shows, especially compared with the 4-mer end motif rankings for plasma nuclear DNA of the first patient and the second patient as illustrated in FIG. 15A, the 4-mer end motif rankings for pleural fluid nuclear DNA of the first patient and the second patient are less correlated. The 4-mer end motif rankings for pleural fluid nuclear DNA of the first patient and the second patient had a Pearson's r of 0.832.

The correlation difference may be of interest. Amounts of end motifs may vary because pleural fluid from different patients may have different nuclease profiles. For example, as pleural fluid samples from different patients have certain intrinsic property or inherent mechanisms, the ranking of pleural fluid samples from different patients tends to show a less correlated relationship for different patients than plasma samples for different patients.

FIG. 16A shows motif O/E ratios for plasma nuclear DNA of two patients. The two patients both have medical conditions causing pleural effusion. In particular, x-axis represents the motif O/E ratio of 4-mer end motifs in plasma samples for the first patient, whereas y-axis represents the motif O/E ratio of 4-mer end motifs in plasma samples for the second patient. As FIG. 16A shows, the O/E ratio of 4-mer end motifs of plasma nuclear DNA for the first patient and the second patient are highly correlated, with a Pearson's r of 0.998.

FIG. 16B shows motif O/E ratios for pleural fluid nuclear DNA of two patients. In some embodiments, the two patients both have medical conditions causing pleural effusion. In particular, x-axis represents the motif O/E ratio of 4-mer end motifs in pleural fluid samples for the first patient, whereas y-axis represents the motif O/E ratio of 4-mer end motifs in pleural fluid samples for the second patient. As FIG. 16B shows, especially compared with the motif O/E ratios for plasma nuclear DNA of the first patient and the second patient as illustrated in FIG. 16A, the motif O/E ratios for pleural fluid nuclear DNA of the first patient and the second patient are less correlated, with a Pearson's r of 0.832.

Similar correlation patterns are also found across all seven patients with medical conditions causing pleural effusion, as shown in correlation matrices.

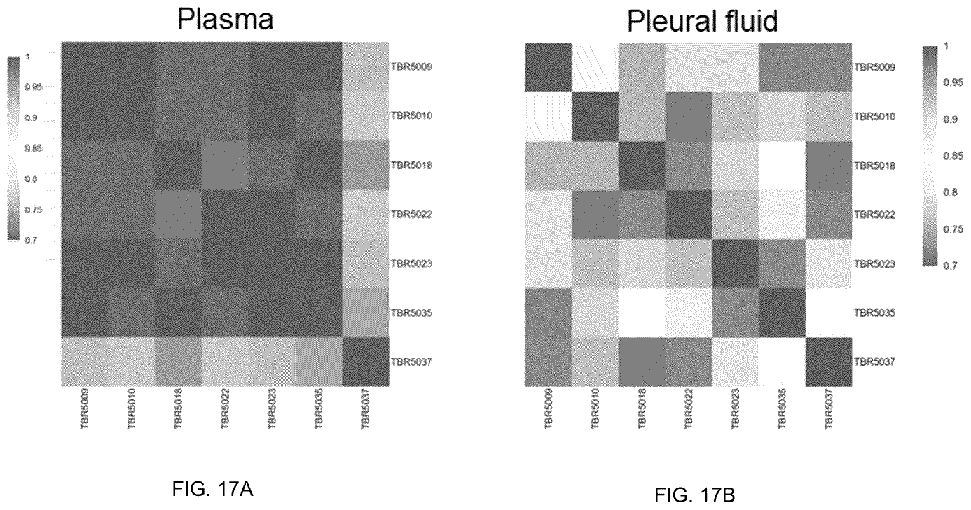

FIG. 17A shows a correlation matrix representing the correlation coefficients for plasma samples of seven patients. In this example, the correlation coefficient is Pearson's r, although other types of correlation values may be used. As FIG. 17A illustrates, the correlation coefficients for plasma samples among the seven patients are generally in a range of 0.9 to 1.

FIG. 17B shows a correlation matrix representing the correlation coefficients for pleural fluid samples according to embodiments of the present disclosure. As FIG. 17B illustrates, the correlation coefficients for pleural fluid samples among the seven patients are generally in a range of 0.7 to 1. FIG. 17B shows a general decrease in correlation compared to FIG. 17A.

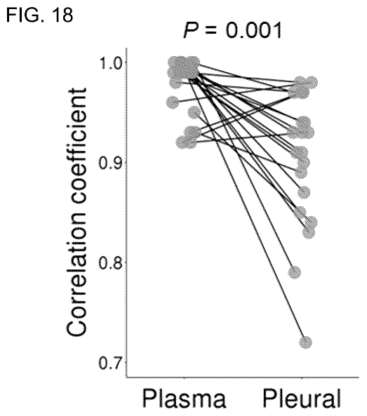

FIG. 18 shows a comparison of correlation coefficients for plasma and pleural fluid samples according to embodiments of the present disclosure. As FIG. 18 illustrates, the correlation coefficients among different pleural fluid samples were significantly lower than correlation coefficients among different plasma samples, which may indicate that the end motif profiles of cfDNA in pleural fluid are more variable than those in plasma samples.

### B. End Motif Analysis of MTBC and Host DNA in Pleural Fluid

We also analyzed the end motifs of MTBC DNA in pleural fluid.

### 1. End Motif Correlation Analysis Using O/E Ratio

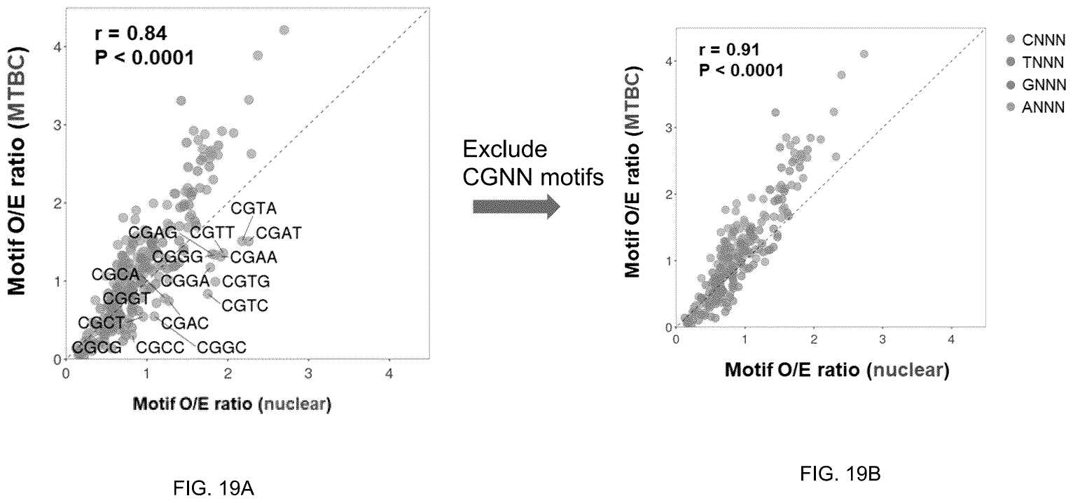

FIG. 19A shows the correlation between human nuclear DNA and MTBC DNA in terms of O/E ratio of 4-mer end motifs for a TB-positive patient. The MTBC DNA in this section refer to those that were aligned to the non-masked MTB genome. The human nuclear DNA does not include mitochondrial DNA, but either or both could be used. Thus, reference to nuclear DNA below can equally apply to mitochondrial DNA or a combination of both. The analysis is based on the end motif profiles of human nuclear DNA and MTBC DNA in a pleural fluid sample of one confirmed TB-positive sample.

As shown in FIG. 19A, the end motif profiles of MTBC DNA were highly correlated with those of nuclear DNA, which has Pearson's r value of 0.84. Some data points (e.g., CGNN motifs) are exceptions to the highly correlated relationship. Based on previous studies, it is known that, in the human genome, the CG methylation is about 60-70%, while in the bacteria the CG methylation level is quite low. Furthermore, previous study also shows that DNASE1L3 has a high preference for the CG when the CG is methylated. Accordingly, it is expected that CG motif is preferred in the human DNA but not the MTBC. Thus, to increase the correlation, excluding CG motifs may further increase the correlation.

FIG. 19B shows a correlation between human nuclear DNA and MTBC DNA in terms of O/E ratio of 4-mer end motifs (excluding CGNN motifs) for a TB-positive patient. As discussed above, although the methylation at the CG site may affect the motif, such methylation at the CG site only affects the motif in the human nuclear DNA but not the MTBC DNA. Accordingly, when determining the correlation between the human nuclear DNA versus the MTBC DNA, excluding the CGNN motifs increases the correlation.

As shown by FIG. 19B, compared with FIG. 19A, the correlation was increased by excluding CGNN motifs, which improves the Pearson's r value from 0.84 to 0.91. Since CpG methylation is rare in bacteria (Phelan et al. Sci Rep. 2018; 8 (1): 160), the preference for CGNN motifs was not observed in MTBC DNA. Therefore, there exists a weaker correlation in the cleavage preferences of CGNN motifs between human nuclear DNA and MTBC DNA given the difference in the overall methylation of the two species.

Additionally, focusing the analysis on the 2-mer analysis broadens the detection coverage. For example, when the analysis focuses on 4-mer end motifs, there may be a total of 256 different kinds of 4-mer end motifs. Meanwhile. for 2-mer end motifs, there may be only 16 end motifs. Accordingly, if 100 MTBC fragments can be detected and 4-mer end motifs are being analyzed, certain 4-mer end motifs may not have coverage and the value would be zero for these 4-mer end motifs. On the other hand, when 2-mer end motifs are being analyzed, most of them could have a value. And for some patients, including the non-TB patients, the number of TB reads may be quite low. If only very limited number of TB reads is available, the analysis would be influenced to have a high noise. Accordingly, to take away the high noise, analysis may be focused on 2-mer end motifs.

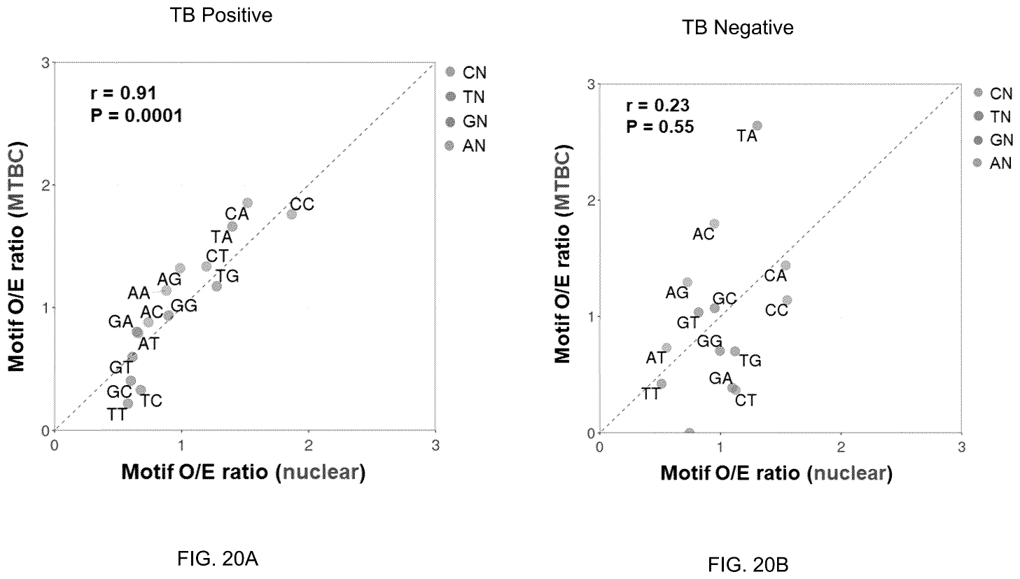

FIG. 20A shows a correlation between human nuclear DNA and MTBC DNA in terms of O/E ratio of 2-mer end motifs (CG motifs excluded) for a TB-positive patient. As shown in FIG. 20A, the 2-mer end motif O/E ratios of MTBC DNA were highly correlated with those of nuclear DNA in the TB-positive sample (Pearson's r=0.91).

FIG. 20B shows a correlation between human nuclear DNA and MTBC DNA in terms of O/E ratio of 2-mer end motifs (CG motifs excluded) for a TB-negative patient. Unlike the correlation between human nuclear DNA and MTBC DNA in terms of O/E ratio of 2-mer end motifs for a TB-positive patient, the correlation between MTBC DNA and the human nuclear DNA is relatively weak in the TB-negative sample (Pearson's r=0.23). Accordingly, a correlation coefficient alone may be used to distinguish TB samples from non-TB samples.

To confirm a correlation coefficient may be used to distinguish TB samples from non-TB samples, we analyzed twenty patients. Among the twenty patients, six patients have TB infection and fourteen patients have no TB infection. Since 2-mer end motif analysis is less susceptible to noise stemming from low sequencing depth, it could be applied to samples with low number of MTBC fragments. The performance of the correlation analysis of 2-mer end motifs is shown below.

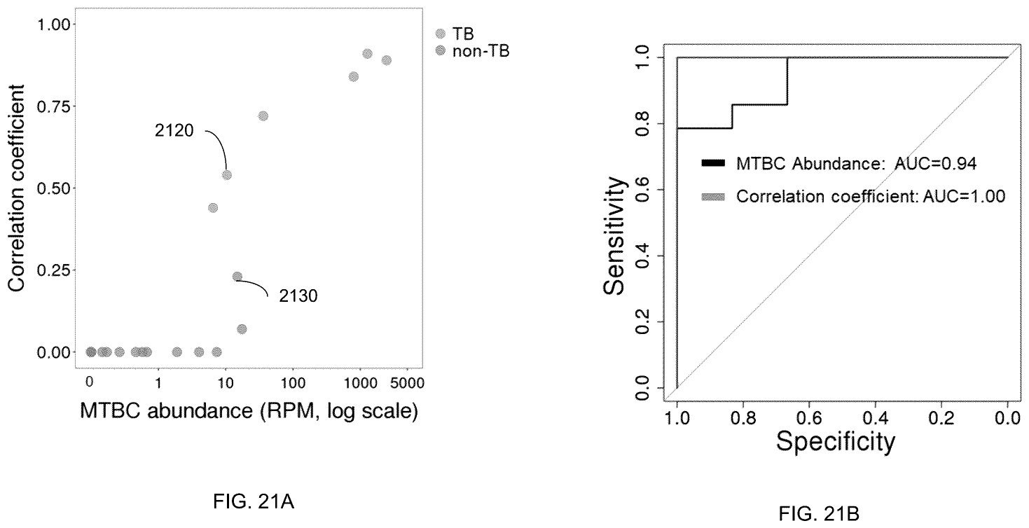

FIG. 21A is a 2-dimensional plot illustrating the correlation coefficient (between human nuclear DNA and MTBC DNA) and MTBC abundance in pleural fluid samples according to embodiments of the present disclosure. The x-axis is the MTBC abundance and the y-axis is the correlation coefficient. Higher value means high correlation. The red dots 2120 are from the TB cases and the blue dots 2130 are from non-TB cases. By using the different correlation coefficients shown by TB cases and non-TB cases, the two groups (e.g., TB vs. non-TB) can be distinguished. For example, a cutoff any value between about 0.25 and 0.4 provides perfect separation between the TB and non-TB cases.

Although the correlation of FIG. 21A is determined based on the O/E ratio (e.g., as shown in FIGS. 20A and 20B), different parameters may be used. For example, a frequency (e.g., a relative percentage of each end motif) or ranking analysis (e.g., of a frequency or O/E ratio) may be used for each end motif instead of O/E ratio. As part of comparing the nuclear DNA and MTB DNA, to determine a correlation value, a clustering analysis may be conducted between end motif profiles of the nuclear DNA and the MTBC DNA for a TB detection.

The measured parameter (e.g., frequency, O/E ratio, or ranking of such value) of the nuclear DNA and the MTBC DNA can form a pair of vectors for which a pairwise comparison is performed. For instance, a distance can be determined between the two vectors, which can be treated as multidimensional data points. The determination of a correlation value can be implicit via a use of a machine learning model, e.g., clustering, PCA, SVM, or neural networks. Such models can receive both sets of values and provide a score (e.g., probability of a microbial disease) that is dependent on the correlation between the end motif profiles of human DNA and microbial DNA. Thus, samples can be assigned scores based on amounts of end motifs for the human DNA and microbial DNA fed into an ML model, where the scores show the difference between TB cases and non-TB cases.

FIG. 21B is a ROC analysis showing the performance of abundance and correlation coefficient in distinguishing TB samples from non-TB samples according to embodiments of the present disclosure. The analysis shows that the correlation coefficient has an AUC of 1.0 and the MTBC abundance using a non-masked genome has an AUC of 0.94. Thus, the correlation coefficient improves the accuracy.

### 2. End Motif Frequency

The data in the previous section used O/E ratio. The data in this section used end motif frequency, which was determined as an amount of ending sequences having a particular end motif divided by the total number of end motifs determined from the cell-free DNA fragments. The data shows that end motif frequency can also be used.

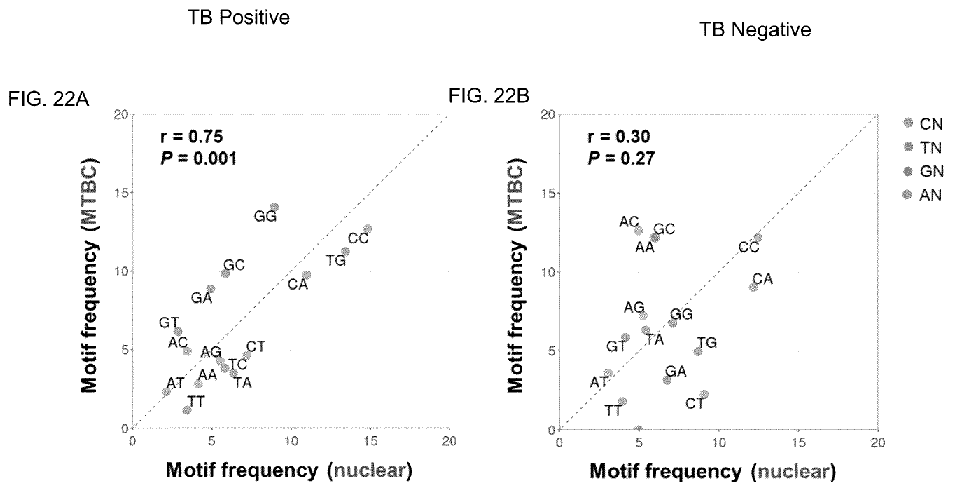

FIG. 22A shows a correlation between human nuclear DNA and MTBC DNA for a TB-positive patient in terms of frequency of 2-mer end motifs (CG excluded). As shown in FIG. 22A, for a TB-positive sample, the frequencies of 2-mer end motifs of MTBC DNA are highly correlated with those of nuclear DNA (Pearson's r=0.75).

FIG. 22B shows a correlation between human nuclear DNA and MTBC DNA for a TB-negative patient in terms of frequency of 2-mer end motifs (CG excluded). As shown in FIG. 22B, compared with the correlation between human nuclear DNA and MTBC DNA for a TB-positive patient in terms of frequency of 2-mer end motifs, the correlation between MTBC DNA and human nuclear DNA is relatively weak in the TB-negative sample (Pearson's r=0.30).

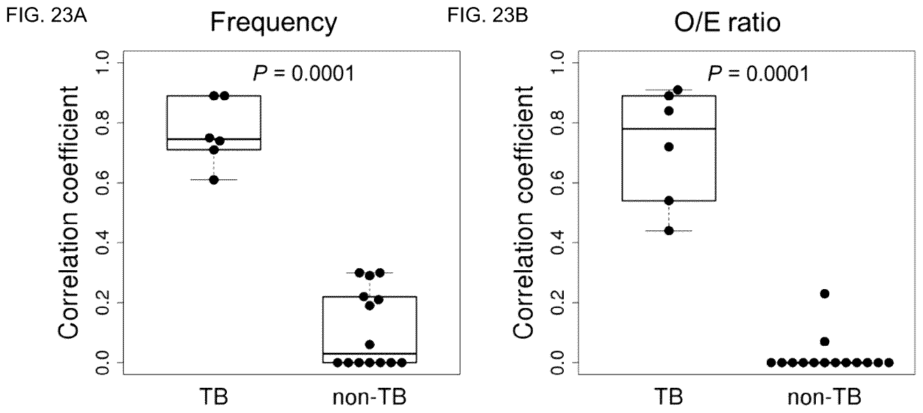

FIG. 23A shows a comparison of correlation coefficients of frequencies between 20 TB and non-TB group samples. The performance (e.g., effective separation of TB samples and non-TB samples) of correlation analysis by using 2-mer motif frequencies confirms that the correlation coefficients of frequencies can be used to distinguish TB samples from non-TB samples. As shown, a cutoff anywhere in the range of about 0.3 to 0.55 provides a perfect discrimination between TB and non-TB.

FIG. 23B shows a comparison of correlation coefficients of O/E ratios between the 20 TB and non-TB group samples according to embodiments of the present disclosure. The performance (e.g., effective separation of TB samples and non-TB samples) of correlation analysis by using 2-mer motif frequencies confirms that the correlation coefficients of O/E ratios can be used to distinguish TB samples from non-TB samples.

### 3. PCA Analysis and Machine Learning Models

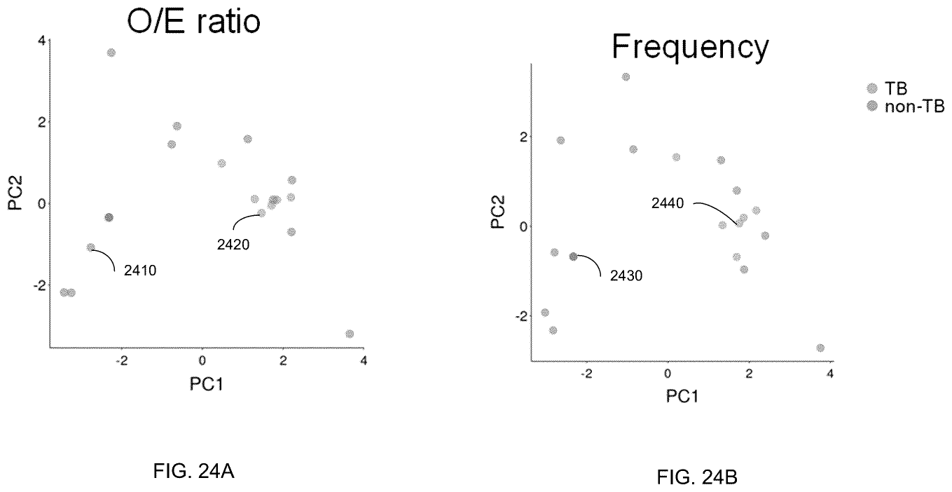

FIG. 24A shows a principal components analysis (PCA) of 2-mer end motif O/E ratio (excluding CG motifs) of MTBC DNA. As FIG. 24A provides, there is a moderate clustering of the TB 2420 and non-TB 2410 groups. FIG. 24B shows a principal components analysis (PCA) of 2-mer end motif frequency (excluding CG motifs) of MTBC DNA. Similar as FIG. 24A, FIG. 24B shows a moderate clustering of the TB 2440 and non-TB 2430 groups. More extensive clustering may occur using more components.

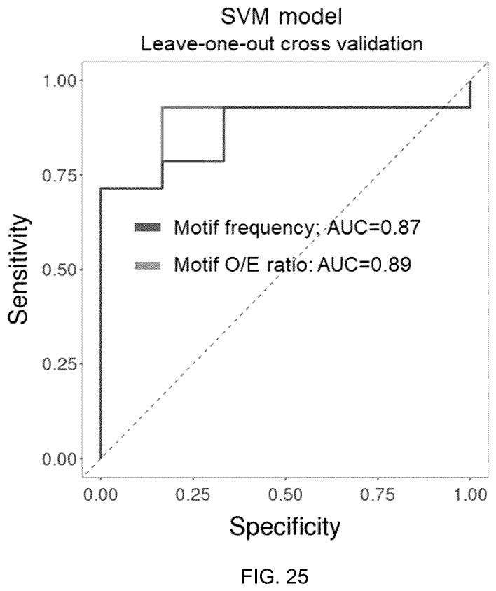

FIG. 25 provides ROC curves showing the performance of machine learning models trained on MTBC 2-mer motif frequency or motif O/E ratio in distinguishing TB samples from non-TB samples according to embodiments of the present disclosure. As illustrated by FIG. 25, the machine learning models described herein show good performance with an AUC of 0.89 for motif O/E ratio and an AUC of 0.87 for motif frequency.

Besides MTBC 2-mer motif frequency or motif O/E ratio, the machine learning models may also be trained on different parameters or any k-mer motif (e.g., 3-mer motif, 4-mer motif, etc.) as desired. In some embodiments, the machine learning models may include a support vector machine (SVM) model, e.g., using a leave one out cross validation.

As examples, an input to the machine learning (ML) models may be abundance values, O/E ratios, frequencies, and the like of a target microbial species and optionally host DNA (e.g., a human nuclear DNA). The input may also include a correlation value between or among the input parameters, which may be determined separately from the ML model. The output of the machine learning model may be such a correlation value among the input values. In some embodiments, the correlation may not be a single value.

Accordingly, various embodiments can determine the level of a particular microbial disease using only end motifs of microbial DNA but can also use end motifs of host DNA. A method can analyze a biological sample to determine a level of a particular microbial disease in the biological sample of a subject, where the biological sample includes cell-free DNA of microbes. The method can include analyzing cell-free DNA molecules from the biological sample to obtain sequence reads. Analyzing a cell-free DNA molecule can include determining an end sequence motif of at least one end of the cell-free DNA molecule; identifying, by comparing the sequence reads to a microbial reference genome, a first group of the cell-free DNA molecules as being from a particular microbial species that is associated with the particular microbial disease; determining, using the sequence reads of the first group of the cell-free DNA molecules, a first amount for each of a set of end sequence motifs of the first group of the cell-free DNA molecules, thereby obtaining first amounts; and determining a classification of the level of the particular microbial disease for the subject using the first amounts. As shown in FIGS. 24A-25B, this can be done by inputting the first amounts into a machine learning model that provides a probability of a sample having the disease or not. The higher probability (possibly required to be above a threshold) can be used to determine the classification.

### C. Fragment Size of MTBC DNA Fragments

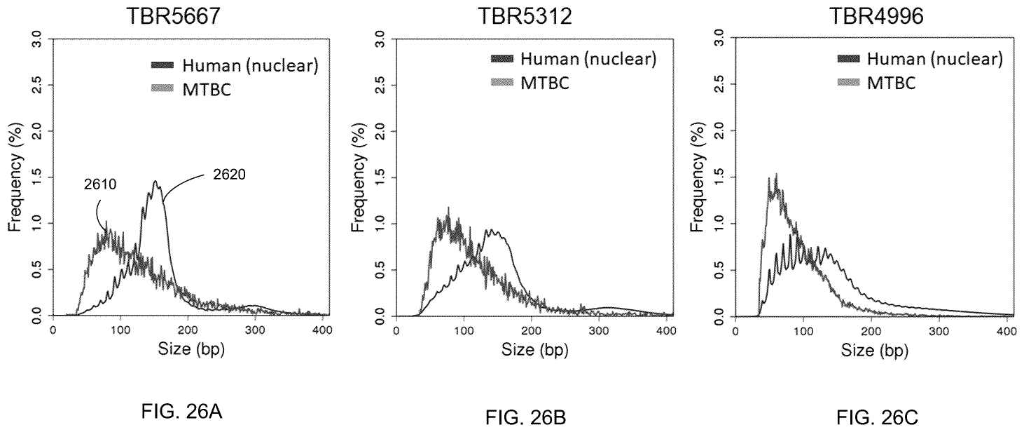

FIGS. 26A-26C illustrate fragment size distribution of human nuclear DNA 2620 (blue) and MTBC 2610 (red) in TB samples. As shown in FIGS. 26A-26C, the MTBC DNA tends to be shorter than human nuclear DNA. For example, as illustrated by FIG. 26A, the MTBC DNA has a median fragment size of 113 bp, whereas the human nuclear DNA has a median fragment size of 149 bp.

### D. Method Using Correlation of End Motifs

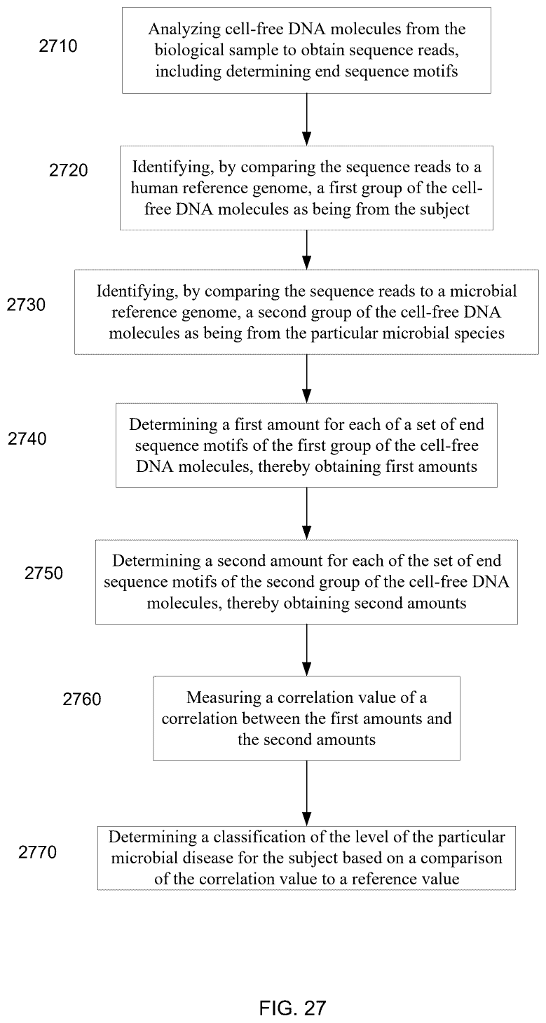

FIG. 27 is a flowchart illustrating a method for analyzing a biological sample to determine a level of a particular microbial disease in the biological sample of a subject according to embodiments of the present disclosure.

At block 2710, cell-free DNA molecules from the biological sample are analyzed to obtain sequence reads. Block 2710 can be performed in a similar manner as block 1210 of method 1200. Analyzing a cell-free DNA molecule can include determining an end sequence motif of at least one end of the cell-free DNA molecule.

At block 2720, a first group of the cell-free DNA molecules is identified as being from the subject by comparing the sequence reads to a human reference genome. Such alignment (mapping) can be performed using various software tools, as described herein and will be appreciated by the skilled person. The first group of the cell-free DNA molecules from the subject can include mitochondrial DNA and/or nuclear DNA. Accordingly, at least a portion of the first group of the cell-free DNA molecules identified as being from the subject can include nuclear DNA.

At block 2730, a second group of the cell-free DNA molecules is identified as being from a particular microbial species that is associated with the particular microbial disease. The identification can be performed by comparing the sequence reads to a microbial reference genome, which may or may not be a masked microbial reference genome.

In some embodiments, the second group of the cell-free DNA molecules can be identified by comparing the sequence reads to multiple microbial reference genomes in a taxonomy tree, where the multiple microbial reference genomes include the microbial reference genome. Additionally or alternatively, the second group of the cell-free DNA molecules can be identified by comparing the sequence reads to the microbial reference genome using alignment software that outputs a mapping quality. A sequence read can be identified as being from the particular microbial species when the mapping quality is greater than a threshold.

At block 2740, a first amount is determined for each of a set of end sequence motifs of the first group of the cell-free DNA molecules, thereby obtaining first amounts. Determining the first amount can use the sequence reads of the first group of the cell-free DNA molecules. The first amounts may be absolute values or normalized values, e.g., a relative frequency, such as a percentage of the first group that has a particular end sequence motif. The normalization can account for the sequence context of the reference genome used, e.g., an O/E ratio.

As examples, the set of end sequence motifs can be of length two bases (2-mers), three bases (3-mers), or four bases (4-mers). The set of end sequence motifs can exclude a CG end motif, as described herein. As examples, the set of end sequence motifs can include at least 10, 11, 12, 13, 14, 15, 16, 64, or 256 end sequence motifs.

At block 2750, a second amount is determined for each of the set of end sequence motifs of the second group of the cell-free DNA molecules, thereby obtaining second amounts. Determining the second amount can use the sequence reads of the second group of the cell-free DNA molecules. The second amounts may also be absolute values or normalized values, e.g., as described for the first amounts.

The first amounts and the second amounts can be a ratio of an observed amount and an expected amount, referred to as O/E herein. For example, an expected amount of the set of end sequence motifs can be determined based on a reference sequence of the human reference genome. Then, determining the classification can include normalizing each of the first amounts with the expected amount to obtain normalized first amounts that are used to measure the correlation value.

As a further example, any of such values can be used to determine a ranking, which can be used as the first amount. Accordingly, the first amount can be a ranking of each of the set of end sequence motifs based on an abundance of the first group of the cell-free DNA molecules having a respective end sequence motif of the set. Similarly, the second amount can be a ranking of each of the set of end sequence motifs based on an abundance of the second group of the cell-free DNA molecules having a respective end sequence motif of the set.

At block 2760, a correlation value of a correlation between the first amounts and the second amounts is measured. Measuring the correlation value can include determining a difference between a respective first amount and a respective second amount for each of the set of end sequence motifs. The differences can be aggregated and potentially normalized by the number of end motifs in the set.

In some embodiments, the correlation value can be the Pearson correlation coefficient (r), which measures linear correlation. It is a number between −1 and 1 that measures the strength and direction of the relationship between two variables. When one variable changes, the other variable changes in the same direction. Such a correlation can be measured as the ratio between the covariance of two variables and the product of their standard deviations. The Pearson correlation coefficient could be calculated by using the formula below, where r is correlation coefficient, xᵢ are a set of values of the first variable, x̅ is the mean of values of the first variable, yᵢ are a set of values of the second variable, y̅ is the mean of values of the second variable.

$$r = \frac{Σ ( x i - \overline{x} ) ( y i - \overline{y} )}{\sqrt{{Σ ( {x}_{i} - \overline{x} )}^{2} {Σ ( {y}_{i} - \overline{y} )}^{2}}}$$

The correlation values include but are not limited to Pearson correlation coefficient, Spearman's rank correlation, Phi correlation, Kendall rank correlation, Jaccard similarity, Cosine similarity etc.

At block 2770, a classification of the level of the particular microbial disease for the subject is determined based on a comparison of the correlation value to a reference value. The reference value can be selected using measurements obtained from one or more reference samples for which a classification is known, e.g., disease positive or disease negative. For instance, the reference value can be determined using a first cohort of training samples from subjects known to have the particular microbial disease and a second cohort of training samples from subjects known to not have the particular microbial disease. FIG. 21A shows a plot with measurements for such reference samples. An example reference value could be between 0.25 and 0.4.

In some embodiments, a machine learning model can be used to measure the correlation value and determine the classification of the level of the particular microbial disease for the subject. For example, the first amounts and the second amounts can be input to the machine learning model, which can determine the correlation value as an intermediate step prior to outputting a classification, which may be a probability.

As with method 1200, the particular microbial species can be a bacterial species, such as Mycobacterium tuberculosis complex (MTBC). The particular microbial disease can be tuberculosis.

### VI. NANOPORE EXAMPLES

We also performed nanopore sequencing using the above techniques. To verify the feasibility of Nanopore platform for detection of mycobacterial DNA in pleural fluid samples, we selected 10 MTB target captured libraries, which were sequenced on Illumina platform as well. Two of the samples were either culture or qPCR confirmed positive for TB infection (TB group), and the other 8 samples were culture or qPCR negative (non-TB). We sequenced the libraries in one Nanopore PromethION run, which produced an average of 6.9 million raw sequencing reads per sample.

### A. Masked MTBC Genome

For the masking technique, we first aligned the raw sequencing reads to a human reference genome. The unaligned non-human reads were used to detect MTBC reads by using two different strategies. (1) Align the non-human reads to human and MTB reference genome using bowtie2 without the proposed masking strategy. (2) Align the non-human reads to human and masked MTB reference genome using bowtie2.

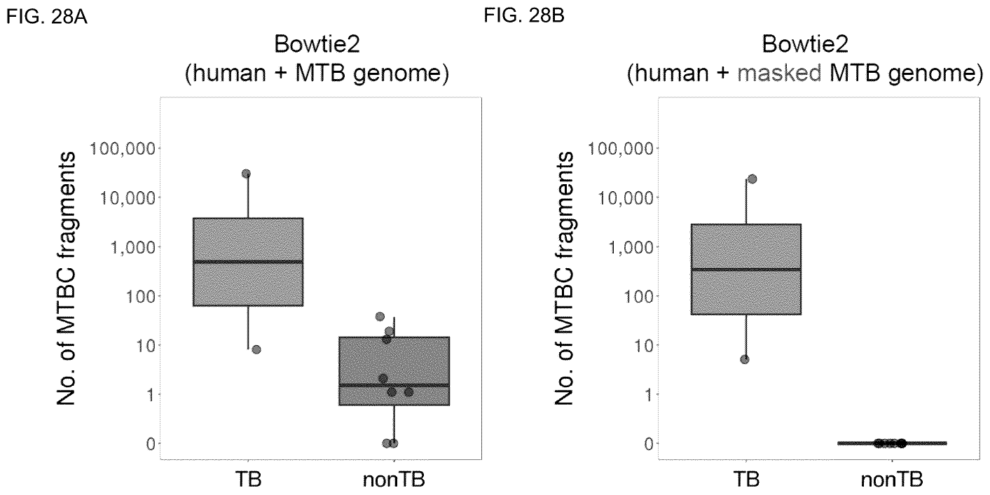

FIGS. 28A-28B show the number of MTBC DNA fragments detected in TB-positive (TB) and TB-negative (non-TB) samples by using two different alignment methods (FIG. 28A) without masking (FIG. 28B) with masking. FIG. 28A shows a plot of the number of MTBC fragments determined using nanopore sequencing without masking the MTB reference genome for TB and non-TB subjects. FIG. 28B shows a plot of the number of MTBC fragments determined using nanopore sequencing and masking the MTB reference genome for TB and non-TB subjects.

As shown, MTBC sequences were successfully detected in the two TB-positive samples using two strategies. However, only by using the second strategy (FIG. 28B) with masking, MTBC reads were not detected in any of the TB-negative (non-TB) samples. And the separation between TB and non-TB is complete (FIG. 28B), no overlap with 100% sensitivity and specificity while the first strategy had overlap between TB and non-TB subject, which would result in a less than 100% sensitivity and/or specificity. Using the masking strategy, a reference value (cutoff value) anywhere between 0 and about 5 provides 100% accuracy.

### B. End Motifs

The TB-positive sample with the highest number of MTBC reads was used to perform the analysis below.

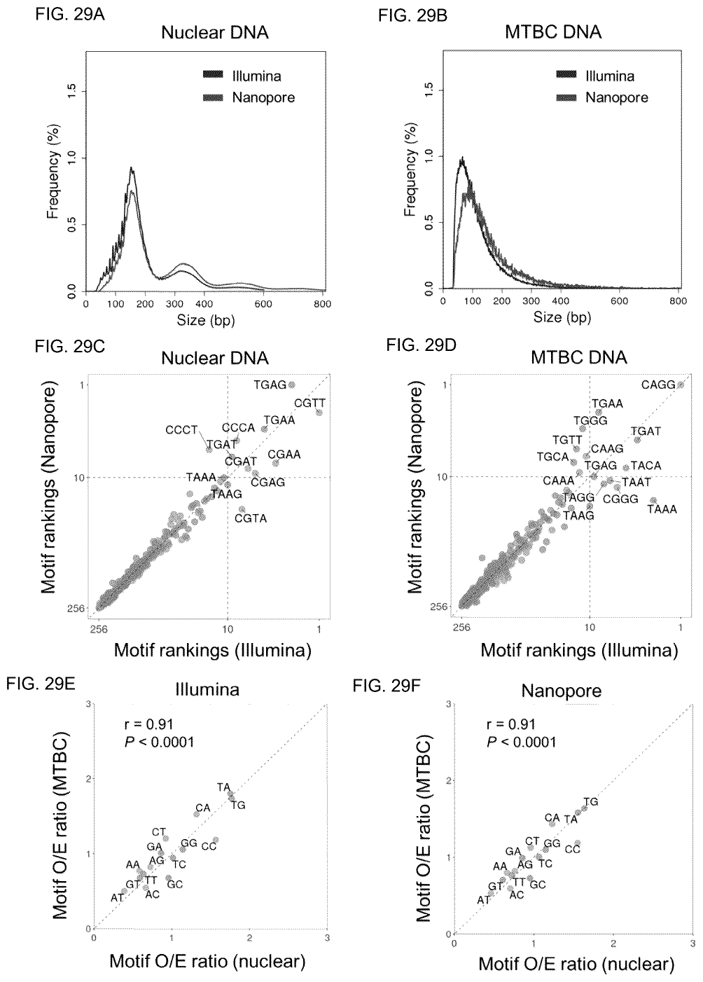

FIGS. 29A-29F show the comparison of Nanopore and Illumina in terms of size and end motif analysis. FIG. 29A shows the size distribution of nuclear DNA in pleural fluid. FIG. 29B shows the size distribution of MTBC DNA in pleural fluid. In general, the size distribution is similar, except that the Nanopore data provides more long DNA fragments than Illumina, for both nuclear and MTBC cfDNA.

FIG. 29C shows the rankings of end motifs by O/E ratios of nuclear DNA. FIG. 29D shows the rankings of end motifs by O/E ratios of MTBC DNA. The end motif profiles determined from data of both Illumina and Nanopore are highly correlated, for both nuclear and MTBC DNA.

FIG. 29E shows the correlation between human nuclear DNA and MTBC DNA in terms of O/E ratio of 2-mer end motifs by using Illumina data. FIG. 29F shows the correlation between human nuclear DNA and MTBC DNA in terms of O/E ratio of 2-mer end motifs by using Nanopore data. Both Illumina and Nanopore data show a high correlation of 2-mer end motif between nuclear and MTBC DNA.

Accordingly, such techniques would work across various types of assays.

### VII. EXAMPLE IMPLEMENTATION

Below are example implementation details that can be used with various embodiments of the present disclosure.

### A. Classification of MTBC-Derived Sequencing Reads

The paired-end sequencing reads were aligned to the reference human genome (for example, hg38). Reads that were not aligned to the human genome were re-aligned to a microbial database including complete reference genomes from species of Mycobacteria family and other microbes. The microbial origin of these sequencing reads (i.e., taxonomy) could be determined. The alignment procedure was performed by using Kraken2 (Wood et al. Genome Biol. 2019; 20 (1): 257). The alignment could also be performed by using other bioinformatics algorithms including BLAST, FASTA, Bowtie, BWA, BFAST, SHRIMP, SSAHA2, NovoAlign, SOAP etc., which may be used with a mapping quality threshold to be assigned to any given species or genus and may be used within a taxonomy.

### B. DNA Fragment End Motif Analysis

An end motif determination can be performed based on the length of the k-mer used, for example, a 4-mer motif corresponding to the 4-nucleotide sequence on each 5′ end (Watson and Crick) of DNA molecules. The end motif frequency was defined as the fraction of an end motif over the total number of end motifs.

As the DNA end motif frequencies are affected by the sequence context of reference genome being analyzed, some implementations can normalize by the sequence context of the reference genome. To compare the end preferences across different sequence contexts (for example between human DNA and mycobacterial DNA), we applied a normalization method. End motif counting was performed within a region(s) corresponding to the reference genome (e.g., masked or unmasked microbial or human, which may be masked, e.g., for repeat regions). A sliding window that is the same size as the end motif can be slid across the region to identify and count occurrences of each end motif. For each end motif, an expected frequency E can be determined as the fraction of K-mers from the reference genome that have a particular end motif.

The end motif frequencies measured in a sample were calculated as the fraction of an end motif over the total number of end motifs, which were referred to as expected end motif frequencies. The 5′ end motif frequencies of sequenced DNA fragments were referred to as observed motif frequencies (O). Additionally or alternatively, 3′ end motif frequencies can be used. The observed end motif frequencies were normalized by the expected end motif frequencies (E), the resultant values were defined as O/E ratio. A higher value of O/E ratio indicates a higher preference for the end motifs. Human nuclear cfDNA end motif frequencies and O/E ratios could be determined with the same method.

### C. DNA Fragment Size Analysis

The DNA fragment size could be determined by the number of nucleotides between the outermost genomic coordinates of paired-end sequencing reads of a DNA fragment. The fragment size of human nuclear DNA could be deduced directly from alignment results. For MTBC, the classified MTBC reads would be re-aligned to microbial database by using tools including Bowtie2, BWA or SOAP. Then the size of MTBC fragments would be determined.

### VIII. EXAMPLE TREATMENTS

Embodiments may further include treating a subject after determining a classification of a level of infection for the subject. For example, treatment can be provided according to a predicted amount of microbes in the biological sample of the subject. In some instances, the treatment is provided based on a type of tissue at which the infection has occurred. The tissue type can be used to guide antibiotic treatment, antibiotic specific for resistant strains, a surgery, or any other form of treatment. And the level of infection can be used to determine how aggressive to be with any type of treatment, which may also be determined based on the level of disease. For example, sepsis may be treated by an antibiotic treatment and blood pressure support drugs. In some embodiments, the more the value of a parameter (e.g., amount or size) exceeds the reference value, the more aggressive the treatment may be.

Example treatments for treating the microbial infection includes but are not limited to the following: antibiotics or antibacterials (possibly specific for resistant strains); antivirals; antiparasitic agents; and antifungals. In some instances, different types of drugs and treatments are provided based on a type of microbe species identified from the subject. For example, if Mycobacterium tuberculosis is found in the subject, drugs such as Isoniazid (INH), rifampin (RIF), rifabutin, rifapentine (RPT), pyrazinamide (PZA), or any fluoroquinolone can be provided. In another example, if Clostridium botulinum bacteria is identified in the subject, antitoxins can be provided.

### IX. EXAMPLE SYSTEMS

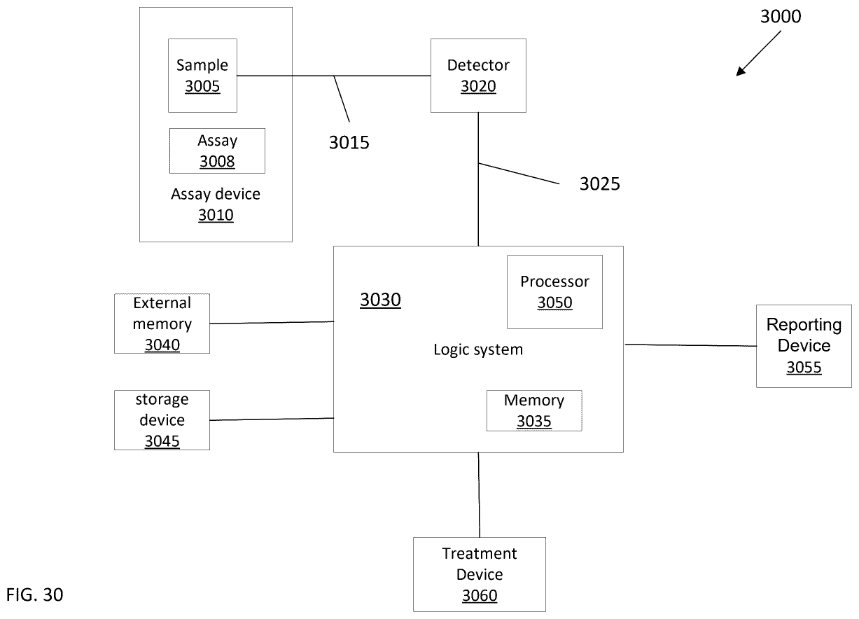

FIG. 30 illustrates a measurement system 3000 according to an embodiment of the present disclosure. The system as shown includes a sample 3005, such as cell-free nucleic acid molecules (e.g., DNA and/or RNA of a host and/or of microbes) within an assay device 3010, where an assay 3008 can be performed on sample 3005. For example, sample 3005 can be contacted with reagents of assay 3008 to provide a signal of a physical characteristic 3015 (e.g., sequence information of a cell-free nucleic acid molecule). An example of an assay device can be a flow cell that includes probes and/or primers of an assay or a tube through which a droplet moves (with the droplet including the assay). Physical characteristic 3015 (e.g., a fluorescence intensity, a voltage, or a current), from the sample is detected by detector 3020. Detector 3020 can take a measurement at intervals (e.g., periodic intervals) to obtain data points that make up a data signal. In one embodiment, an analog-to-digital converter converts an analog signal from the detector into digital form at a plurality of times.

Assay device 3010 and detector 3020 can form an assay system, e.g., a sequencing system that performs sequencing according to embodiments described herein. A data signal 3025 is sent from detector 3020 to logic system 3030. As an example, data signal 3025 can be used to determine sequences and/or locations in a reference genome of nucleic acid molecules (e.g., DNA and/or RNA). Data signal 3025 can include various measurements made at a same time, e.g., different colors of fluorescent dyes or different electrical signals for different molecule of sample 3005, and thus data signal 3025 can correspond to multiple signals. Data signal 3025 may be stored in a local memory 3035, an external memory 3040, or a storage device 3045. The assay system can be comprised of multiple assay devices and detectors.

Logic system 3030 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 3030 and the other components may be part of a stand-alone or network connected computer system, or they may be directly attached to or incorporated in a device (e.g., a sequencing device) that includes detector 3020 and/or assay device 3010. Logic system 3030 may also include software that executes in a processor 3050. Logic system 3030 may include a computer readable medium storing instructions for controlling measurement system 3000 to perform any of the methods described herein. For example, logic system 3030 can provide commands to a system that includes assay device 3010 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 3000 may also include a treatment device 3060, which can provide a treatment to the subject. Treatment device 3060 can determine a treatment and/or be used to perform a treatment. Examples of such treatment can include surgery, radiation therapy, chemotherapy, immunotherapy, targeted therapy, hormone therapy, and stem cell transplant. Logic system 3030 may be connected to treatment device 3060, e.g., to provide results of a method described herein. The treatment device may receive inputs from other devices, such as an imaging device and user inputs (e.g., to control the treatment, such as controls over a robotic system).

Measurement system 3000 may also include a reporting device 3055, which can present results of any of the methods describe herein, e.g., as determined using the measurement system. Reporting device 3055 can be in communication with a reporting module within logic system 3030 that can aggregate, format, and send a report to reporting device 3055. The reporting module can present information determined using any of the method described herein. The information can be presented by reporting device 3055 in any format that can be recognized and interpreted by a user of the measurement system 3000. For example, the information can be presented by reporting device 3055 in a displayed, printed, or transmitted format, or any combination thereof.

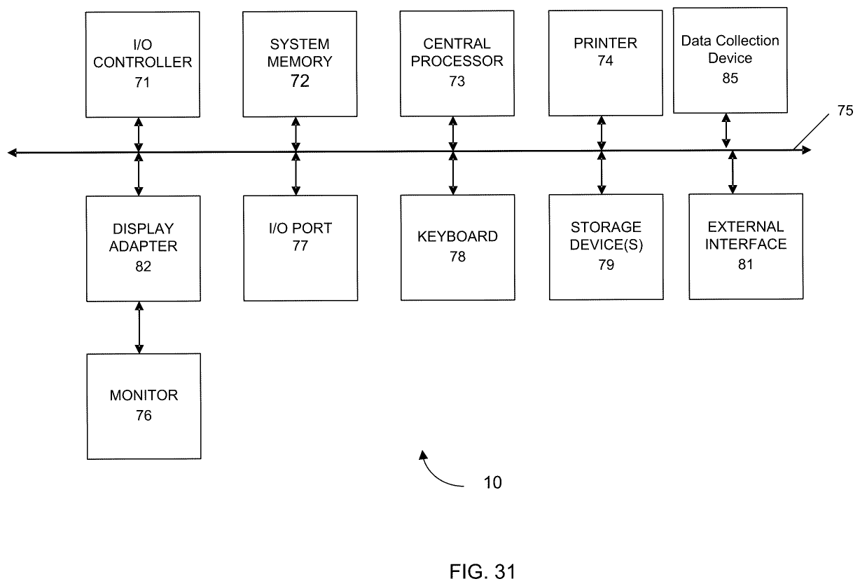

Any of the computer systems mentioned herein may utilize any suitable number of subsystems. Examples of such subsystems are shown in FIG. 31 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 31 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

What is claimed is:

1. A method of analyzing a biological sample to determine a level of a particular microbial disease in the biological sample of a subject, the biological sample including cell-free DNA of microbes and cell-free DNA of the subject, the method comprising:
analyzing cell-free DNA molecules from the biological sample to obtain sequence reads;
storing a masked microbial reference genome of a particular microbial species that is associated with the particular microbial disease, wherein the masked microbial reference genome is generated from a microbial reference genome of the particular microbial species, the microbial reference genome including (1) specific regions that are identified as unique to the particular microbial species and (2) non-specific genomic regions that are shared with one or more other species, and wherein the masked microbial reference genome is generated by removing the non-specific genomic regions from the microbial reference genome;
aligning the sequence reads to the masked microbial reference genome to identify a group of the cell-free DNA molecules as being from the particular microbial species;
determining an amount of the group of the cell-free DNA molecules; and
determining a classification of the level of the particular microbial disease for the subject based on a comparison of the amount to a reference value.

2. The method of claim 1, further comprising:
identifying the non-specific genomic regions that are shared with the one or more other species by comparing the microbial reference genome of the particular microbial species to one or more other reference genomes of the one or more other species.

3. The method of claim 2, wherein identifying the non-specific genomic regions includes:
partitioning the one or more other reference genomes into a set of K-mers, wherein K is between 20 and 35; and
aligning the set of K-mers to the microbial reference genome to identify the non-specific genomic regions.

4. The method of claim 3, wherein the non-specific genomic regions correspond to a subset of the set of K-mers that aligned to the microbial reference genome.

5. The method of claim 2, wherein identifying the non-specific genomic regions includes:
partitioning the microbial reference genome into a set of K-mers, wherein K is between 20 and 35; and
aligning the set of K-mers to the one or more other reference genomes to identify the non-specific genomic regions.

6. The method of claim 5, wherein the non-specific genomic regions correspond to a subset of the set of K-mers that aligned to the one or more other reference genomes.

7. The method of claim 5, wherein the non-specific genomic regions are identified as regions not corresponding to a subset of the set of K-mers that did not align to the one or more other reference genomes.

8. The method of claim 1, wherein the sequence reads are aligned to multiple microbial reference genomes in a taxonomy tree to identify the group of the cell-free DNA molecules as being from the particular microbial species, and wherein the multiple microbial reference genomes include the masked microbial reference genome.

9. The method of claim 1, wherein aligning the sequence reads to the masked microbial reference genome uses alignment software that outputs a mapping quality, and wherein a sequence read is identified as being from the particular microbial species when the mapping quality is greater than a threshold.

10. The method of claim 1, wherein the amount is normalized.

11. The method of claim 9, wherein the amount is normalized by dividing by a total number of the sequence reads or by a number of the sequence reads that are from a genome of the subject.

12. The method of claim 1, wherein the one or more other species includes the subject.

13. A method of analyzing a biological sample to determine a level of a particular microbial disease in the biological sample of a subject, the biological sample including cell-free DNA of microbes and cell-free DNA of the subject, the method comprising:
analyzing cell-free DNA molecules from the biological sample to obtain sequence reads, wherein analyzing a cell-free DNA molecule includes:
determining an end sequence motif of at least one end of the cell-free DNA molecule;
identifying, by comparing the sequence reads to a human reference genome, a first group of the cell-free DNA molecules as being from the subject;
identifying, by comparing the sequence reads to a microbial reference genome, a second group of the cell-free DNA molecules as being from a particular microbial species that is associated with the particular microbial disease;
determining, using the sequence reads of the first group of the cell-free DNA molecules, a first amount for each of a set of end sequence motifs of the first group of the cell-free DNA molecules, thereby obtaining first amounts;
determining, using the sequence reads of the second group of the cell-free DNA molecules, a second amount for each of the set of end sequence motifs of the second group of the cell-free DNA molecules, thereby obtaining second amounts;
measuring a correlation value of a correlation between the first amounts and the second amounts; and
determining a classification of the level of the particular microbial disease for the subject based on a comparison of the correlation value to a reference value.

14. The method of claim 13, wherein the second group of the cell-free DNA molecules are identified by comparing the sequence reads to multiple microbial reference genomes in a taxonomy tree, and wherein the multiple microbial reference genomes include the microbial reference genome.

15. The method of claim 13, wherein the microbial reference genome is a masked microbial reference genome.

16. The method of claim 13, wherein the second group of the cell-free DNA molecules are identified by comparing the sequence reads to the microbial reference genome uses alignment software that outputs a mapping quality, and wherein a sequence read is identified as being from the particular microbial species when the mapping quality is greater than a threshold.

17. The method of claim 13, wherein at least a portion of the first group of the cell-free DNA molecules identified as being from the subject include mitochondrial DNA.

18. The method of claim 13, wherein at least a portion of the first group of the cell-free DNA molecules identified as being from the subject include nuclear DNA.

19. The method of claim 13, wherein the first amount is a relative frequency of an end sequence motif.

20. The method of claim 13, further comprising:
determining an expected amount of the set of end sequence motifs based on a reference sequence of the human reference genome, wherein determining the classification includes normalizing each of the first amounts with the expected amount to obtain normalized first amounts that are used to measure the correlation value.

21. The method of claim 13, wherein the first amount is a ranking of each of the set of end sequence motifs based on an abundance of the first group of the cell-free DNA molecules having a respective end sequence motif of the set, and wherein the second amount is a ranking of each of the set of end sequence motifs based on an abundance of the second group of the cell-free DNA molecules having a respective end sequence motif of the set.

22. The method of claim 13, wherein the set of end sequence motifs are of length two bases, three bases, or four bases.

23. The method of claim 22, wherein the set of end sequence motifs exclude a CG end motif.

24. The method of claim 22, wherein the set of end sequence motifs include at least 10 end sequence motifs.

25. The method of claim 13, wherein measuring the correlation value includes determining a difference between a respective first amount and a respective second amount for each of the set of end sequence motifs.

26. The method of claim 13, wherein a machine learning model is used to measure the correlation value and determine the classification of the level of the particular microbial disease for the subject.

27. The method of claim 26, wherein the first amounts and the second amounts are input to the machine learning model.

28. The method of claim 1, wherein the reference value is determined using a first cohort of training samples from subjects known to have the particular microbial disease and a second cohort of training samples from subjects known to not have the particular microbial disease.

29. The method of claim 1, wherein the particular microbial species is a bacterial species.

30. The method of claim 29, the bacterial species is Mycobacterium tuberculosis complex (MTBC).

31. The method of claim 29, wherein the particular microbial disease is tuberculosis.

32. The method of claim 1, wherein analyzing the cell-free DNA molecules includes receiving sequence reads obtained from targeted sequencing of the cell-free DNA molecules from the biological sample.

33. The method of claim 32, further comprising performing the targeted sequencing.

34. The method of claim 32, wherein the targeted sequencing uses capture probes for the microbial reference genome that are at a higher concentration than capture probes for a human reference genome.

35. A computer product comprising a non-transitory computer readable medium storing a plurality of instructions that, when executed, cause a computer system to perform a method of analyzing a biological sample to determine a level of a particular microbial disease in the biological sample of a subject, the biological sample including cell-free DNA of microbes and cell-free DNA of the subject, the method comprising:
analyzing cell-free DNA molecules from the biological sample to obtain sequence reads;
storing a masked microbial reference genome of a particular microbial species that is associated with the particular microbial disease, wherein the masked microbial reference genome is generated from a microbial reference genome of the particular microbial species, the microbial reference genome including (1) specific regions that are identified as unique to the particular microbial species and (2) non-specific genomic regions that are shared with one or more other species, and wherein the masked microbial reference genome is generated by removing the non-specific genomic regions from the microbial reference genome;
aligning the sequence reads to the masked microbial reference genome to identify a group of the cell-free DNA molecules as being from the particular microbial species;
determining an amount of the group of the cell-free DNA molecules; and
determining a classification of the level of the particular microbial disease for the subject based on a comparison of the amount to a reference value.
